(19) United States
(12) Patent Application Publication
(10) Pub. No.: US 2022/0151544 A1
(43) Pub. Date: May 19, 2022

(54) DUAL POSITION SCANNER AND INTEGRATED SYSTEM FOR SKIN CANCER MAPPING AND TRACKING

(71) Applicant: MARPE TECHNOLOGIES LTD., Katzrin (IL)

(72) Inventors: Yaakov NAVON, Katzrin (IL); Tovi BACHAR, Haifa (IL)

(21) Appl. No.: 17/435,838

(22) PCT Filed: Mar. 1, 2020
(86) PCT No.: PCT/IL2020/050232

§ 371 Date: Sep. 2, 2021

Related U.S. Application Data
(60) Provisional application No. 62/813,054, filed on Mar. 3, 2019.

(51) Int. Cl. A61B 5/00

(52) U.S. Cl. CPC A61B 5/444; A61B 5/742; A61B 5/748; A61B 5/0064

(57) ABSTRACT

A system for mapping and tracking at least one mole on a surface of a body comprises: an optical system configured to scan a surface of the body and to generate at least one image of at least a portion of the surface and including at least one primary imaging device attachable to at least one first maneuvering mechanism and a switching mechanism in mechanical communication with the at least one optical system. The switching mechanism is configured to reversibly switch the at least one optical system between generating the at least one image of a standing patient and generating the at least one image of a patient lying down.

### FIELD OF THE INVENTION

The present invention generally pertains to a simple, fast and accurate system and method for identifying and tracking skin cancer by mapping and tracking skin lesions such as moles based on scanning, with a patient either lying down or standing. The entire body area can be digitally mapped and automatically tracked for moles and other skin lesions.

### BACKGROUND OF THE INVENTION

Skin cancers and melanoma in particular are serious life-threatening types of cancer. Morbidity and mortality rates for melanoma are well documented by the National Cancer Institute—over 73,870 new cases and about 9,940 deaths from melanoma were reported in the United States in 2015. Early detection can lead to cure in almost 99% of cases, while late detection leads to less than 15% survival. Achieving an early diagnosis by using a computerized body surface scan can lead to early treatment, vastly improving the chances of recovery.

The current practice of full body skin examination is carried by inspecting visually mole by mole over the entire body. Suspicious moles are thoroughly checked and biopsied when necessary. This process is very slow, time consuming and thus very expensive. Moreover, and clinically this is of the most importance, moles are hardly ever tracked over time, as there is no efficient record keeping system. An additional problem is that the lack of dermatologists carrying out full body skin examinations creates a bottleneck. As a result, the number of tests in western countries is negligible: less than one percent of the population is routinely checked.

Current devices and methods deal with two type of skin test—devices that image the entire body, showing all moles, or devices that analyze single suspicious moles. Current solutions suffer from the following problems: a complex photography process, low image quality, insufficient automation of mole analysis and an inefficient process, which increases expenditure of time by the dermatologist, and the lack of a web-based solution.

The Melafind® (http://www.melafind.com/) system analyzes single moles. It looks below the skin's surface to objectively evaluate clinically atypical pigmented skin lesions and classify them based on the level of 3-dimensional morphological disorganization from under the skin. The system is intended for use by dermatologists only, and the atypical pigmented skin lesions must be identified by the dermatologist before the Melafind® system is deployed.

The MoleExpert® software system (http://moleexpert.com/) offers computer aided mole mapping, professional mole mapping software, automated detection of new or changed lesions, and dynamic zoom for high resolution images. Two similar follow-up images of a relevant body site for one patient are compared. MoleExpert macro automatically detects nevi in the first and in the follow-up image and computes the mole mapping. Additionally the software extracts features relevant to the size, shape, and brightness of each lesion and compares these features for all mapped nevi. Extracted moles are shown in full resolution providing a detailed image of the nevus.

The same company also offers the FotoFinder bodystudio, a device to provide whole-body scan. With the FotoFinder bodystudio total body mapping can be performed in only 10 minutes. The system displays on-screen the body area to be photographed and the corresponding patient pose. The laptop and camera are in permanent connection offering an optimized imaging workflow where the software takes control of the entire imaging process. Captured images are displayed immediately on-screen and directly stored in the patient database. The camera slides up and down the stand for fast and consistent photography of all body areas. The bodystudio cart features the unique Laser Liner for consistent patient positioning at baseline and follow-ups: The laser displays a red line on the floor to show the patient where to position. It also assures consistent distance between camera and patient, even when you move the system from room to room. Total body mapping cannot get any easier.

For follow-up images all camera settings are automatically restored. FotoFinder's Ghost Feature displays the baseline photo in the background of the live image and assists the user for consistent patient-positioning. Consistent follow-up images are taken every time by any user.

In addition, the company offers the FotoFinder dermoscope, which combines the MoleExpert software with the FotoFinder bodystudio to offer all in one, dermoscopic skin cancer screening and mole mapping. The system automatically links digital dermoscopic images of moles to an overview image, making the identification and tracking of lesions easy. Through on-screen comparison of overview and microscopic mole images at follow-up visits, diagnostic accuracy can be significantly increased as even the slightest changes in mole structure are visualized and melanoma in situ and melanomas that do not satisfy the classical clinical features of melanoma are easier detected, examined and specified for surgery. All of these are intended to be used by medical personnel.

The Skin Scan SRL (https://skinvision.com/) is intended to be used by laypersons. It is an IOS app designed to keep track of the size and shape of moles. The software is loaded onto a smartphone. The user then photographs moles using the phone's built-in camera. The photographed moles are then uploaded to a server, where it is analyzed. The software can also track changes in the size and shape of a mole over time, by comparing pictures of the mole taken at different times. The software also tells the user a time interval between reanalyses of the mole and allows the user to set a reminder to perform the reanalysis.

Techniques to analyze single moles include confocal microscopy, an optical imaging technique used to increase optical resolution and contrast of a micrograph by using point illumination and a spatial pinhole to eliminate out-of-focus light in specimens that are thicker than the focal plane. It enables the reconstruction of three-dimensional structures from the obtained images. The technique uses point illumination and a pinhole in an optically conjugate plane in front of the detector to eliminate out-of-focus signal, thereby improving the resolution of the image. As only one point in the sample is illuminated at a time, 2D or 3D imaging requires scanning over a regular raster in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples.

However, all of these techniques require long scanning times, can require long processing times to join overlapping images from independent 2D cameras into a single image, it can be difficult to get simultaneously into proper alignment and proper focus all of a fairly large number of 2D camera, and patient movement can make it difficult to ensure both that the proper mole is later imaged in close up and that images remain in proper alignment and proper focus during imaging. It can be difficult to ensure that the correct mole is selected for further imaging at a later time. In addition, since images must be made of the bare skin, patients can suffer considerable discomfort during imaging when virtually all of the skin is exposed, both by the embarrassment of being nearly nude and by the physical discomfort caused.

Furthermore, the present systems require a considerable amount of physicians' time as all examinations must be carried out by the physician, and the present systems are not configured to allow remote access to data, preventing both remote working by the physician and consulting with other physicians who may be physically remote both from the primary physician and the patient.

Furthermore, there are some disagreements between experts as to which is the better position in which to carry out the scan—standing or lying down. Advantages of standing position scanning are that it requires less floor space and that the scanner can be positioned in a corner of the room when not in use. On the other hand, taking dermoscopic images is less comfortable for the physician when a patient is standing rather than lying down and scanning a patient lying down requires more floor space. It is therefore a long felt need to provide a system that requires neither long scanning times nor long processing times, that does not require the patient to be in a fixed orientation (standing or lying), that allows at least some data collection and at least some data analysis by technicians and that allows safe communication of data to remote sites electronically.

### SUMMARY OF THE INVENTION

It is an object of the present invention to disclose a system for increasing the throughput of total body skin tests and enabling thorough tracking of moles.

It is another object of the invention to disclose a system for mapping and tracking at least one mole on a surface of a body, comprising:
at least one optical system configured to scan a surface of said body and to generate at least one image of at least a portion of said surface, said optical system comprising at least one primary imaging device attachable to at least one first maneuvering mechanism, said at least one image comprising substantially all of said portion of said surface facing said at least one primary imaging device, said at least one mole identifiable from said at least one image; and
at least one switching mechanism in mechanical communication with said at least one optical system,
wherein said at least one switching mechanism is configured to reversibly switch said at least one optical system between generating said at least one image of a standing patient and generating said at least one image of a patient lying down.

It is another object of the invention to disclose the system as described above, additionally comprising at least one measurement device configured to make at least one three dimensional (3D) measurement of at least one dimension of said body.

It is another object of the invention to disclose the system as described above, additionally comprising at least one secondary imaging device attachable to at least one second maneuvering mechanism, said second maneuvering mechanism configured to automatically aim said secondary imaging device at said at least one mole, said secondary imaging device thereupon being configured to automatically capture at least one image of said at least one mole.

It is another object of the invention to disclose the system as described above, wherein, by correlation between a scanned image comprising an image of at least one image mole and at least one 3D measurement of at least one corresponding body mole corresponding to said at least one image mole, said secondary imaging device is accurately aimable at said at least one corresponding body mole.

It is another object of the invention to disclose the system as described above, additionally comprising at least one bi-directional light pointer in communication with an indicating mechanism, said bi-directional light pointer having at least two modes, said at least two modes selected from a group consisting of: a display-to-body mode, a body-to-display mode and any combination thereof.

It is another object of the invention to disclose the system as described above, wherein, in said display-to-body mode, at least one displayed mole in a displayed image being selected using an indicating mechanism in communication with a display, light from said light pointer is shined on a corresponding at least one body mole on said surface.

It is another object of the invention to disclose the system as described above, wherein said indicating mechanism is selected from a group consisting of: a touch-sensitive display, a symbol movable over the image, moving of an image to a predetermined location, and any combination thereof,

It is another object of the invention to disclose the system as described above, wherein, in said body-to-display mode, at least one body mole on said surface being selected by manually pointing said light pointer at said at least one body mole, an indication is provided for a corresponding at least one display mole in a displayed image.

It is another object of the invention to disclose the system as described above, wherein indication of the corresponding display mole is selected from a group consisting of: overlaying a symbol on said image of said corresponding display mole, surrounding said image of said corresponding display mole by a symbol, moving said image of said corresponding display mole to a predetermined position in a display, and any combination thereof.

It is another object of the invention to disclose the system as described above, wherein said predetermined position is a center of said display.

It is another object of the invention to disclose the system as described above, wherein said manually pointing of said light pointer is via a member of a group consisting of: of a joystick in communication with said light pointer, sensors indicating position and orientation in space of said light pointer, and any combination thereof.

It is another object of the invention to disclose the system as described above, additionally comprising a hair alignment mechanism configured to orient said hair substantially perpendicular to said surface of said body facing said at least one primary imaging device, to minimize apparent size of said hair in said image.

It is another object of the invention to disclose the system as described above, wherein a start of alignment of said hair is either simultaneous with a start of said imaging of at least a portion of said body's surface or before a start of said imaging of at least a portion of said body's surface, said alignment of said hair continuing during said imaging of said at least a portion of said body's surface.

It is another object of the invention to disclose the system as described above, wherein said primary imaging device is a one dimensional (1D) camera.

It is another object of the invention to disclose the system as described above, wherein said first maneuvering mechanism can maneuver said at least one primary imaging device so as to cause it to move in a manner selected from a group consisting of: moving in the X direction, moving in the Y direction, moving in the Z direction, rotating about an axis in the X direction, rotating about an axis in the Y direction, rotating about an axis in the Z direction, and any combination thereof.

It is another object of the invention to disclose the system as described above, wherein said at least one primary imaging device keeps in focus said surface of said body facing said at least one primary imaging device by at least one of: maneuvering said at least one primary imaging device in said Z direction and refocusing said at least one primary imaging device.

It is another object of the invention to disclose the system as described above, wherein said at least one primary imaging device acquires images in said X direction of substantially the entire said surface of said body facing said at least one primary imaging device by at least one of: moving said at least one primary imaging device in at least said X direction, rotating said at least one primary imaging device about said Y direction, and providing a at least one primary imaging device with a field of view at least as wide as said surface of said body facing said at least one primary imaging device.

It is another object of the invention to disclose the system as described above, wherein said at least one primary imaging device acquires images in said Y direction of substantially the entire said surface of said body facing said at least one primary imaging device by at least one of: maneuvering said at least one primary imaging device in said Y direction, and rotating said at least one primary imaging device about said X direction.

It is another object of the invention to disclose the system as described above, additionally comprising an alignment synchronization mechanism configured to synchronize movement of said alignment mechanism and said maneuvering mechanism such that said hair alignment mechanism aligns hairs in portions of said body being imaged.

It is another object of the invention to disclose the system as described above, wherein said first maneuvering mechanism additionally comprises at least one light source configured to illuminate at least a portion of said body, said at least one light source configured to provide sufficient light such that clear images of said body can be acquired.

It is another object of the invention to disclose the system as described above, wherein said at least one light source is configured to illuminate said at least a portion of said body's surface in a manner selected from a group consisting of: a homogeneous manner and a heterogeneous manner; wherein, for said heterogeneous manner of illumination, the difference between the illumination of at least one part of said illuminated portion of said body's surface and at least one other part of said illuminated portion of said body's surface being configured to optimize contrast in said image.

It is another object of the invention to disclose the system as described above, wherein said first maneuvering mechanism additionally comprises at least one sensor configured to indicate distance between said at least one primary imaging device and said body's surface so as to enable said at least one primary imaging device to provide clear images of said body's surface.

It is another object of the invention to disclose the system as described above, wherein said system additionally comprises at least one processing system in communication with said at least one primary imaging device, comprising a program of machine-readable instructions embodied on a computer readable memory and executable by a digital data processor, configured to combine said at least one image to form a single image of substantially the entire surface of said body.

It is another object of the invention to disclose the system as described above, wherein said at least one processing system additionally comprises a program of machine-readable instructions embodied on a computer readable memory and executable by a digital data processor, configured to locate in said at least one image said at least one mole.

It is another object of the invention to disclose the system as described above, wherein said at least one processing system additionally comprises a program of machine-readable instructions embodied on a computer readable memory and executable by a digital data processor, configured to map a location of said at least one mole on said surface.

It is another object of the invention to disclose the system as described above, wherein said at least one processing system additionally comprises a program of machine-readable instructions embodied on a computer readable memory and executable by a digital data processor, configured to create at least one image of said at least one mole.

It is another object of the invention to disclose the system as described above, wherein said at least one processing system additionally comprises a program of machine-readable instructions embodied on a computer readable memory and executable by a digital data processor, configured to identify, from said at least one mole, a set of suspicious moles, said set being empty if no suspicious mole is present on said surface.

It is another object of the invention to disclose the system as described above, wherein at least one secondary imaging device is configured to generate, said set of suspicious moles not being empty, at least one close-up image of at least one member of said set of suspicious moles.

It is another object of the invention to disclose the system as described above, wherein said at least one processing system is additionally in communication with said at least one secondary imaging system, and additionally comprises a program of machine-readable instructions embodied on a computer readable memory and executable by a digital data processor, configured to instruct at least one of a group consisting of: said at least one primary imaging device and said at least one secondary imaging system to move in a manner selected from a group consisting of: move along said X direction, move along said Y direction, move along said Z direction, rotate around said X direction, rotate around said Y direction, and rotate around said Z direction.

It is another object of the invention to disclose the system as described above, wherein said at least one processing system in communication with said at least one secondary imaging system additionally comprises a program of machine-readable instructions embodied on a computer readable memory and executable by a digital data processor, configured to instruct said at least one secondary imaging system to generate, said set of suspicious moles not being empty, said at least one close-up image of said at least one member of said set of suspicious moles; and, for at least one image of at least one suspicious mole in said set of suspicious moles, parameterize said at least one suspicious mole from at least one image of said at least one suspicious mole.

It is another object of the invention to disclose the system as described above, wherein said system additionally comprises an image synchronization mechanism configured to synchronize said image taken by said at least one primary imaging device and said first maneuvering mechanism by maneuvering the same.

It is another object of the invention to disclose the system as described above, wherein said body can be re-imaged periodically.

It is another object of the invention to disclose the system as described above, wherein, for each of said at least one moles, at least one image of said at least one mole generated at at least one first time is compared to at least one image of said at least one mole generated at least one second time, from said comparison the evolution of said at least one mole over time being trackable.

It is another object of the invention to disclose the system as described above, additionally comprising a distinguishing system for enabling distinguishing between hair and skin, said distinguishing system comprising at least one of: an infrared camera, a static electric field and air suction.

It is another object of the invention to disclose the system as described above, wherein said static electric field is generated by an electrostatic generator.

It is another object of the invention to disclose the system as described above, wherein said electrostatic generator is at least one of a van de Graaf generator and a Pelletron generator.

It is another object of the invention to disclose the system as described above, wherein said distinguishing system is synchronized with the maneuvering of said camera, minimizing the area on the body where hairs are affected.

It is another object of the invention to disclose the system as described above, additionally comprising an exposing mechanism configured to expose at least partially a section of said body to said at least one primary imaging device, wherein said section of said body is exposed at such times as it is being imaged, while the rest of said body is maintained covered.

It is another object of the invention to disclose the system as described above, additionally comprising an exposure synchronization mechanism configured to synchronize movement of said exposing mechanism and said first maneuvering mechanism such that said camera images only said exposed section.

It is another object of the invention to disclose the system as described above, wherein said exposed section of said body has width in the Y direction in a range from approximately 2 cm to approximately 3 cm.

It is another object of the invention to disclose the system as described above, wherein said at least one mole is analyzable according to ABCDE parameters, said parameters comprising: (i) measuring one mole according to its (A)symmetry, (B)order shape, (C)olor and (D)iameter, and (ii) displaying said mole's history over a series of said images over time, (E)volution of said at least one mole being tracked and of any new mole being identified and tracked.

It is another object of the invention to disclose the system as described above, wherein, said set of suspicious moles not being empty, said at least one suspicious mole is parameterizable from at least one image selected from a group consisting of: an image of said at least one mole, a close-up image of said at least one mole, and any combination thereof.

It is another object of the invention to disclose the system as described above, wherein, for each said at least one suspicious mole, said parameterization creates a score for each said at least one suspicious mole.

It is another object of the invention to disclose the system as described above, wherein said at least one suspicious mole is grouped according to said score.

It is another object of the invention to disclose the system as described above, wherein said at least one suspicious moles is ranked according to said score.

It is another object of the invention to disclose the system as described above, wherein said at least one suspicious mole is identifiable by at least one selected from a group consisting of: an operator instructs said at least one processing system that a given mole is suspicious, and said at least one processing system automatically identifies a mole as suspicious.

It is another object of the invention to disclose the system as described above, wherein said operator instructs said at least one processing system in at least one manner selected from a group consisting of: touching on a touchscreeen said image of said mole, shining a beam of light on said image of said mole on a screen, shining a beam of light on said mole on said body; overlaying an image symbol on said image of said mole, surrounding said mole with an image symbol, locating said image of said mole at a predetermined location in a display, and entering a unique identifier for said mole.

It is another object of the invention to disclose the system as described above, wherein at least one of said measurement device and a height measuring system in communication with said at least one primary imaging device and said tracking system is configured to maintain at least one of a focal distance of said at least one primary imaging device and a distance between said at least one primary imaging device and said body, maintaining said portion of said body being imaged in focus independent of a thickness of said body at a location of said portion of said body.

It is another object of the invention to disclose the system as described above, additionally comprising an exposing mechanism configured to expose at least partially a section of said body to said at least one primary imaging device.

It is another object of the invention to disclose the system as described above, additionally comprising an exposure synchronization mechanism configured to synchronize movement of said exposing mechanism and said first maneuvering mechanism such that said at least one primary imaging device images only said exposed section

It is another object of the invention to disclose the system as described above, wherein said section of said body is exposed at such times as it is being imaged, the rest of said body being maintained covered

It is another object of the invention to disclose the system as described above, wherein said body is a human body.

It is another object of the invention to disclose a method for mapping and tracking at least one mole on a surface of a body, comprising steps of:
providing a system for mapping and tracking at least one mole on a surface of a body, comprising:
at least one optical system configured to scan a surface of said body and to generate at least one image of at least a portion of said surface, said optical system comprising at least one primary imaging device attachable to at least one first maneuvering mechanism, said at least one image comprising substantially all of said portion of said surface facing said at least one primary imaging device, said at least one mole identifiable from said at least one image; and
at least one switching mechanism in mechanical communication with said at least one optical system;
attaching said at least one primary imaging device to said first maneuvering mechanism;
sequentially imaging substantially all of the surface of said body facing said at least one primary imaging device in at least one image by maneuvering said first maneuvering mechanism so as to move said at least one primary imaging device along said surface of said body facing said at least one primary imaging device;
thereby reversibly switching, via said at least one switching mechanism, between generating said at least one image of a standing patient and generating said at least one image of a patient lying down.

It is another object of the invention to disclose the method as described above, additionally comprising a step of providing at least one measurement device configured to make at least one 3D measurement of at least one dimension of said body.

It is another object of the invention to disclose the method as described above, additionally comprising a step of providing at least one secondary imaging device attachable to at least one second maneuvering mechanism, said second maneuvering mechanism configured to automatically aim said secondary imaging device at said at least one mole, said secondary imaging device thereupon being configured to automatically capture at least one image of said at least one mole.

It is another object of the invention to disclose the method as described above, additionally comprising steps of correlating between a scanned image comprising an image of at least one image mole and at least one 3D measurement of at least one corresponding body mole corresponding to said at least one image mole; and accurately aiming, by use of said correlation, said secondary imaging device at said at least one corresponding body mole.

It is another object of the invention to disclose the method as described above, additionally comprising a step of providing at least one bi-directional light pointer in communication with an indicating mechanism, said bi-directional light pointer having at least two modes, said at least two modes selected from a group consisting of: a display-to-body mode, a body-to-display mode and any combination thereof.

It is another object of the invention to disclose the method as described above, additionally comprising steps of selecting, in said display-to-body mode, at least one displayed mole in a displayed image using an indicating mechanism in communication with a display, and of shining light from said light pointer on a corresponding at least one body mole on said surface.

It is another object of the invention to disclose the method as described above, additionally comprising a step of selecting said indicating mechanism from a group consisting of: a touch-sensitive display, a symbol movable over the image, moving of an image to a predetermined location, and any combination thereof.

It is another object of the invention to disclose the method as described above, additionally comprising steps of selecting, in said body-to-display mode, at least one body mole on said surface by manually pointing said light pointer at said at least one body mole, and of providing an indication for a corresponding display mole in a displayed image.

It is another object of the invention to disclose the method as described above, additionally comprising a step of selecting indication of the corresponding display mole from a group consisting of: overlaying a symbol on said image of said corresponding display mole, surrounding said image of said corresponding display mole by a symbol, moving said image of said corresponding display mole to a predetermined position in a display, and any combination thereof

It is another object of the invention to disclose the method as described above, additionally comprising a step of selecting said predetermined position to be a center of said display.

It is another object of the invention to disclose the method as described above, additionally comprising a step of manually pointing said light pointer via a member of a group consisting of: of a joystick in communication with said light pointer, sensors indicating position and orientation in space of said light pointer, and any combination thereof.

It is another object of the invention to disclose the method as described above, additionally comprising steps of providing a hair alignment mechanism; and of orienting said hair substantially perpendicular to said surface of said body facing said at least one primary imaging device, minimizing apparent size of said hair in said image.

It is another object of the invention to disclose the method as described above, additionally comprising a step of starting said aligning of said hair either simultaneously with the start of said imaging of at least a portion of said body's surface or before the start of said imaging of at least a portion of said body's surface, said aligning of said hair continuing during said imaging of said at least a portion of said body's surface.

It is another object of the invention to disclose the method as described above, additionally comprising a step of selecting indication of the corresponding display mole from a group consisting of: overlaying a symbol on said image of said corresponding display mole, surrounding said image of said corresponding display mole by a symbol, moving said image of said corresponding display mole to a predetermined position in a display, and any combination thereof.

It is another object of the invention to disclose the method as described above, wherein said predetermined position is a center of said display.

It is another object of the invention to disclose the method as described above, additionally comprising a step of manually pointing said light pointer is via a member of a group consisting of: of a joystick in communication with said light pointer, sensors indicating position and orientation in space of said light pointer, and any combination thereof.

It is another object of the invention to disclose the method as described above, additionally comprising a step of selecting said primary imaging device to be a one dimensional (1D) camera.

It is another object of the invention to disclose the method as described above, additionally comprising a step of maneuvering said first maneuvering mechanism in a manner selected from a group consisting of: moving in the X direction, moving in the Y direction, moving in the Z direction, rotating about an axis in the X direction, rotating about an axis in the Y direction, rotating about an axis in the Z direction, and any combination thereof.

It is another object of the invention to disclose the method as described above, additionally comprising a step of keeping in focus said surface of said body facing said at least one primary imaging device by at least one of: maneuvering said at least one primary imaging device in said Z direction and refocusing said at least one primary imaging device.

It is another object of the invention to disclose the method as described above, additionally comprising a step of acquiring said images in said X direction of substantially the entire said surface of said body facing said at least one primary imaging device by at least one of: moving said at least one primary imaging device in at least said X direction, rotating said at least one primary imaging device about said Y direction, and providing a at least one primary imaging device with a field of view at least as wide as said surface of said body facing said at least one primary imaging device.

It is another object of the invention to disclose the method as described above, additionally comprising a step of acquiring said images in said Y direction of substantially the entire said surface of said body facing said at least one primary imaging device by at least one of: maneuvering said at least one primary imaging device in said Y direction, and rotating said at least one primary imaging device about said X direction.

It is another object of the invention to disclose the method as described above, additionally comprising a step of providing an alignment synchronization mechanism configured to synchronize movement of said alignment mechanism and said first maneuvering mechanism such that said hair alignment mechanism aligns hairs in portions of said body being imaged.

It is another object of the invention to disclose the method as described above, additionally comprising a step of providing said first maneuvering mechanism with at least one light source configured to illuminate at least a portion of said body, said at least one light source configured to provide sufficient light such that clear images of said body can be acquired.

It is another object of the invention to disclose the method as described above, additionally comprising a step of configuring said at least one light source to illuminate said at least a portion of said body's surface in a manner selected from a group consisting of: a homogeneous manner and a heterogeneous manner; wherein, for said heterogeneous manner of illumination, the difference between the illumination of at least one part of said illuminated portion of said body's surface and at least one other part of said illuminated portion of said body's surface being configured to optimize contrast in said image.

It is another object of the invention to disclose the method as described above, additionally comprising a step of providing said first maneuvering mechanism with at least one sensor configured to indicate distance between said at least one primary imaging device and said surface so as to enable said at least one primary imaging device to provide at least one clear image of said surface.

It is another object of the invention to disclose the method as described above, additionally comprising a step of providing at least one processing system in communication with said at least one primary imaging device, comprising a program of machine-readable instructions embodied on a computer readable memory and executable by a digital data processor, configured to combine said at least one image to form a single image of substantially the entire surface of said body.

It is another object of the invention to disclose the method as described above, additionally comprising a step of providing said at least one processing system with a program of machine-readable instructions embodied on a computer readable memory and executable by a digital data processor, configured to locate in said at least one image said at least one mole.

It is another object of the invention to disclose the method as described above, additionally comprising a step of providing said at least one processing system with a program of machine-readable instructions embodied on a computer readable memory and executable by a digital data processor, configured to map a location of said at least one mole on said surface.

It is another object of the invention to disclose the method as described above, additionally comprising a step of providing said at least one processing system with a program of machine-readable instructions embodied on a computer readable memory and executable by a digital data processor, configured to create at least one image of said at least one mole.

It is another object of the invention to disclose the method as described above, additionally comprising a step of providing said at least one processing system with a program of machine-readable instructions embodied on a computer readable memory and executable by a digital data processor, configured to identify, from said at least one mole, a set of suspicious moles, said set being empty if no suspicious mole is present on said surface.

It is another object of the invention to disclose the method as described above, additionally comprising a step of providing at least one secondary imaging device, said at least one secondary imaging device configured to generate at least one close-up image of at least one member of said set of suspicious moles.

It is another object of the invention to disclose the method as described above, additionally comprising a step of providing said at least one processing system in communication with said at least one secondary imaging device, and comprising a program of machine-readable instructions embodied on a computer readable memory and executable by a digital data processor, configured to instruct at least one of a group consisting of: said at least one primary imaging device and said at least one secondary imaging device to move in a manner selected from a group consisting of: move along said X direction, move along said Y direction, move along said Z direction, rotate around said X direction, rotate around said Y direction, and rotate around said Z direction.

It is another object of the invention to disclose the method as described above, additionally comprising a step of providing said at least one processing system in communication with said at least one secondary imaging device with a program of machine-readable instructions embodied on a computer readable memory and executable by a digital data processor, configured to instruct said at least one secondary imaging device to generate, said set of suspicious moles not being empty, said at least one close-up image of said at least one member of said set of suspicious moles; and, for at least one image of at least one suspicious mole in said set of suspicious moles, parameterize said at least one suspicious mole from at least one image of said at least one suspicious mole.

It is another object of the invention to disclose the method as described above, additionally comprising a step of providing an image synchronization mechanism configured to synchronize said image taken by said at least one primary imaging device and said first maneuvering mechanism by maneuvering the same.

It is another object of the invention to disclose the method as described above, additionally comprising a step of re-imaging said body periodically.

It is another object of the invention to disclose the method as described above, additionally comprising steps of, for each of said at least one mole, generating at least one first image of said at least one mole at at least one first time, generating at least one second image of said at least one mole at at least one second time, comparing said at least one first image and said at least one second image, and, from said comparison, tracking evolution of said at least one mole over time.

It is another object of the invention to disclose the method as described above, additionally comprising a step of providing a distinguishing system for enabling distinguishing between hair and skin, said distinguishing system comprising at least one of: an infrared camera, a static electric field and air suction.

It is another object of the invention to disclose the method as described above, additionally comprising a step of generating said static electric field by an electrostatic generator.

It is another object of the invention to disclose the method as described above, additionally comprising a step of selecting said electrostatic generator from at least one of a van de Graaf generator and a Pelletron generator.

It is another object of the invention to disclose the method as described above, additionally comprising a step of synchronizing said distinguishing system with the maneuvering of said camera, minimizing the area on the body where hairs are affected.

It is another object of the invention to disclose the method as described above, additionally comprising a step of providing an exposing mechanism configured to expose at least partially a section of said body to said at least one primary imaging device, wherein said section of said body is exposed at such times as it is being imaged, while the rest of said body is maintained covered.

It is another object of the invention to disclose the method as described above, additionally comprising a step of providing an exposure synchronization mechanism configured to synchronize movement of said exposing mechanism and said first maneuvering mechanism such that said camera images only said exposed section.

It is another object of the invention to disclose the method as described above, additionally comprising a step of said exposed section of said body having width in the Y direction in a range from approximately 2 cm to approximately 3 cm.

It is another object of the invention to disclose the method as described above, additionally comprising a step of analyzing said at least one mole according to ABCDE parameters, said parameters comprising: (i) measuring one mole according to its (A)symmetry, (B)order shape, (C)olor and (D)iameter, and (ii) displaying said mole's history over a series of said images over time, (E)volution of said mole being tracked and of any new mole being identified and tracked.

It is another object of the invention to disclose the method as described above, additionally comprising a step of, said set of suspicious moles not being empty, parameterizing said at least one suspicious mole from at least one image selected from a group consisting of: an image of said at least one mole, a close-up image of said at least one mole and any combination thereof.

It is another object of the invention to disclose the method as described above, additionally comprising a step of creating a score for each said at least one suspicious mole from said parameterization of said at least one suspicious mole.

It is another object of the invention to disclose the method as described above, additionally comprising a step of grouping said at least one suspicious mole according to said score.

It is another object of the invention to disclose the method as described above, additionally comprising a step of ranking said at least one suspicious mole according to said score.

It is another object of the invention to disclose the method as described above, additionally comprising a step of identifying said at least one suspicious mole by at least one selected from a group consisting of: an operator instructs said at least one processing system that said mole is suspicious, and said at least one processing system automatically identifies said mole as suspicious.

It is another object of the invention to disclose the method as described above, additionally comprising a step of enabling said operator to instruct said at least one processing system in at least one manner selected from a group consisting of: touching on a touchscreeen said image of said mole, shining a beam of light on said image of said mole on a screen, shining a beam of light on said mole on said body; overlaying an image symbol on said image of said mole, surrounding said mole with an image symbol, locating said image of said mole at a predetermined location in a display, and entering a unique identifier for said mole.

It is another object of the invention to disclose the method as described above, additionally comprising steps of selecting at least one of said measurement device and a height measuring system in communication with said at least one primary imaging device and said tracking system, and maintaining at least one of a focal distance of said at least one primary imaging device and a distance between said at least one primary imaging device and said body to maintain said portion of said body being imaged in focus independent of a thickness of said body at a location of said portion of said body.

It is another object of the invention to disclose the method as described above, additionally comprising a step of selecting said body to be a human body.

### BRIEF DESCRIPTION OF THE FIGURES

In order to better understand the invention and its implementation in practice, a plurality of embodiments will now be described, by way of non-limiting example only, with reference to the accompanying drawings, wherein

FIG. 1A-B illustrates work flow diagrams for the prior art;

FIG. 2A-E schematically illustrates embodiments of devices of the present invention;

FIG. 3A illustrates an embodiment of the device configured to scan a patient lying down;

FIG. 3B illustrates an embodiment of the device configured to scan a standing patient;

FIGS. 4-7 illustrate an embodiment of flow charts of the work flow for the present invention;

FIG. 8 illustrates selecting an area of the body for closer examination, showing the images (front, right side, back, left side) from which said area can be selected.

FIG. 9 depicts a human back, with markings indicating moles identified by the system;

FIG. 10 depicts close-ups of moles;

FIG. 11 depicts a human back, showing the evolution of moles over time;

FIG. 12 depicts the evolution of a mole over time;

FIGS. 13-14 schematically illustrate embodiments of hair alignment systems; and

FIG. 15A-B schematically illustrates an embodiment of an exposing system.

### DETAILED DESCRIPTION OF THE PREFERRED EMBODIEMNTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for tracking and mapping body moles based on scanning, digitally mapping and automatically tracking the entire body area for moles and other skin lesions.

In the figures, similar numbers refer to similar parts throughout.

The term ‘suspicious’ hereinafter refers to a mole which shows a morphology that identifies it as possibly being non-benign. Said morphology normally includes such criteria as an irregular border, significant asymmetry, uneven color, red, green or blue areas, scaling, bleeding, and large size.

According to the present invention Said morphology also includes such criteria as uneven color, red, green or blue areas.

The term ‘approximately’ hereinafter refers to plus or minus 20% of the value.

The term ‘Z direction’ hereinafter refers to a vertical direction. This is normally a direction perpendicular to the floor and is normally perpendicular to the upper surface of the patient support system.

The term ‘Y direction’ hereinafter refers to a direction which is perpendicular to the Z direction and is parallel to the longitudinal axis of the patient support system. It is, therefore, parallel to the floor and parallel to the longitudinal axis of the body undergoing scanning.

The term ‘X direction’ hereinafter refers to a direction which is perpendicular to the Z direction and is perpendicular to the Y direction. It is therefore parallel to the floor and parallel to the shorter side of the upper surface of the patient support system.

The term ‘substantially’ hereinafter refers to at least 75% of the item referred to.

The term ‘approximately’ hereinafter refers to within 25% of the value.

The term ‘effective field of view’ hereinafter refers to the maximum extent visible to the one dimensional (1D) camera in a given direction generateable by combining the camera's field of view and the maneuverability of the camera in the Y direction.

The terms ‘camera’ and ‘imaging system’ hereinafter refer to a system for generating at least one image, preferably of a human body and will be used synonymously.

The system comprises a total body photography system, software for automatic mapping, tracking and evaluation of moles, and an interface and workflow design that minimizes the time needed for the physician to perform the scan and analyze the results. The overall throughput of tests can be increased by factor of up to ten, allowing a significantly increased number of skin tests using the existing medical personnel, and thereby allowing a significant increase in early detection of skin cancer.

The system for mapping and tracking body moles can comprise at least some of the following components:
a. At least one optical device configured to scan a patient and to generate at least one image of at least a portion of the skin of the patient.
   Typically, at least one optical device is a primary camera system comprising at least one camera attachable to a maneuvering mechanism. The primary camera system is configured to sequentially image substantially all of the portion of the surface of the body which is facing the at least one camera in at least one image. The maneuvering mechanism is configured to move the at least one camera along the portion of the surface of the body facing the camera, enabling the at least one camera to image substantially all of the portion of the surface of the body facing the at least one camera, thereby generating at least one image of substantially all of the portion of the surface of the body facing the at least one camera.
   Each at least one camera can be any conventional imaging device. Preferably, at least one camera is a 1D camera capturing a line image of the skin, with the maneuvering system moving the 1D camera(s) linearly along the patient's body. In less-preferred embodiments, the at least one camera generates a smaller image so that the maneuvering system moves the camera(s) in a two-dimensional pattern. In some less-preferred embodiments, at least one camera is a 2D camera configured to image a substantial portion of the patient's body such that only little movement of the maneuvering system is needed.
b. At least one measurement device configured to make at least one three dimensional (3D) measurement of at least one dimension of the patient.
c. At least one switching mechanism in mechanical communication with at least one optical device. Typically, the switching mechanism is configured to reversibly rotate the optical device about at least one axis. The switching mechanism enables switching between generating at least one image of at least a portion of the skin of a standing patient and generating at least one image of at least a portion of the skin a patient lying down.
d. At least one secondary imaging system such as, but not limited to, a dermoscope, attachable to a maneuvering mechanism such as, but not limited to, a robotic arm. Using the maneuvering mechanism, the secondary imaging system can be automatically aimed at at least one mole and can automatically capture at least one image of the at least one mole. Typically, suspicious moles will be imaged. Good correlation between at least one image of at least one mole and a corresponding 3D measurement enables the maneuvering mechanism to be accurately aimed at at least one selected mole.
e. A bi-directional light pointer in communication with an indicating mechanism.
   The light pointer can have a display-to-body mode, a body-to-display mode and any combination thereof.
   In display-to-body mode, at least one mole in a scanned image can be selected using the indicating mechanism. The light pointer will then shine light on the corresponding mole on the patient's body.
   The indicating mechanism can be any conventional means of indicating a location in a displayed image, for non-limiting example, a touch-sensitive display, a symbol movable over the image, moving of an image to a predetermined location, and any combination thereof,
   In body-to-display mode, a mole is selectable by manually pointing the light pointer at a mole on the patient's body. The corresponding mole in a displayed image is then indicated. Indicating of the corresponding mole can be by overlaying a symbol on the corresponding mole, surrounding the corresponding mole by a symbol, moving the corresponding mole to a predetermined position in the display, and any combination thereof.
   Manually pointing the light pointer can be by any conventional means of aiming a light pointer, such as, but not limited to, a joystick in communication with the light pointer, sensors indicating the light pointer's position and orientation in space, and any combination thereof.
f. A hair alignment mechanism configured to orient the hairs substantially perpendicular to the surface of the portion of the body facing the 1D camera, so the apparent size in the image of the hairs is minimized.

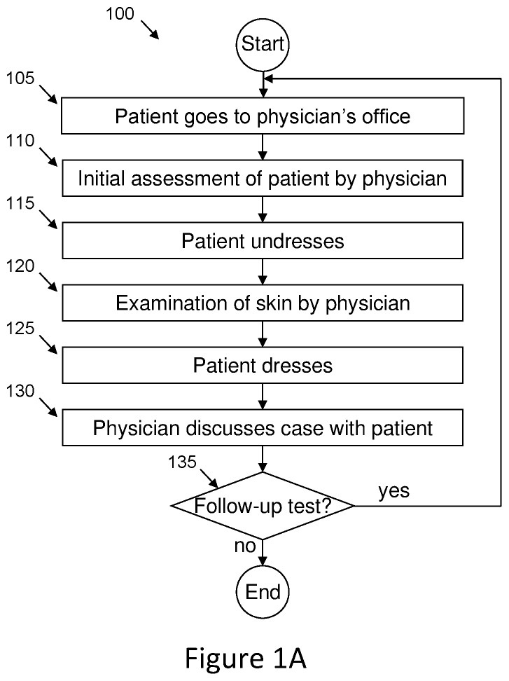

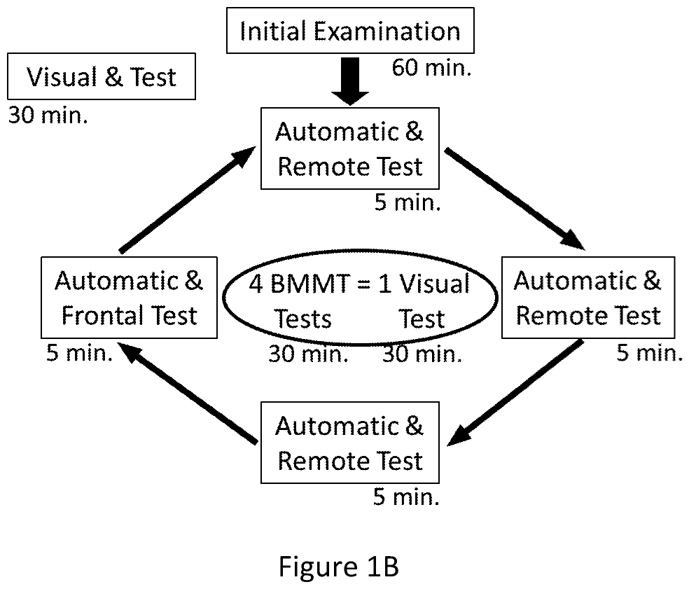

In reference to FIG. 1A-B, a flow chart and work flow diagram are shown for an embodiment of a total body test in the prior art. An flow chart for a total body test (FIG. 1A) comprises the following sequence: an initial assessment of the patient by the physician, the patient removes his clothing and a skin test is performed; the physician examines and photographs the patient's skin. The patient dresses, then the physician discusses the case with him. Instructions for further treatment and follow-up are given at this time. On average, the skin test takes less than 50% of the time; this is not an efficient work flow and throughput is far from optimized. Furthermore, each skin test must follow the same routine; there is no scope for reducing the amount of time each test takes, either for the physician or the patient.

In FIG. 1B, a work flow diagram for the total body test is shown. The initial examination will take about an hour. The automatic and frontal test takes about 15 minutes, while the automatic and remote tests take about 5 minutes each, for a total of 30 minutes of tests out of the total of 60 minutes of the physician's time.

In this disclosure, a new system and method are disclosed with improve throughput and increase the efficiency with which skin tests can be performed. It provides high quality total body images; the close-up images, of suspicious moles, can be acquired by skilled technician rather than by a physician or specialist dermatologist. Although the patient will still need to visit the physician for consultations, there is no need for the patient to meet his dermatologist after each test, thereby reducing the need to visit the physician's office, thereby saving the physician's time. Furthermore, the at least partial automation of the diagnosis of suspicious moles, and the ability to transmit all needed data from the test location to the physician allows computerized diagnosis by the dermatologist, which can substitute for can substitute for some face-to-face meetings with the patient, allows telemedicine and significantly eases getting a second opinion.

The system comprises a total body photography system, software for automatic mapping, tracking and evaluation of moles, and an interface and workflow design that minimizes the time needed for the physician to perform the scan and analyze the results. The overall throughput of tests can be increased by factor of up to ten, allowing a significantly increased number of skin tests using the existing medical personnel, and thereby allowing a significant increase in early detection of skin cancer.

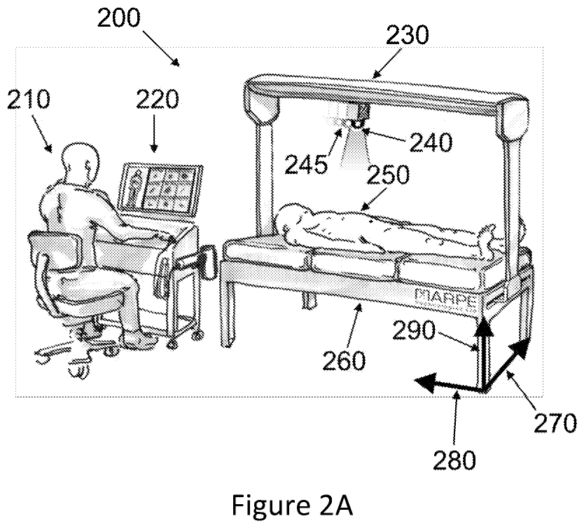

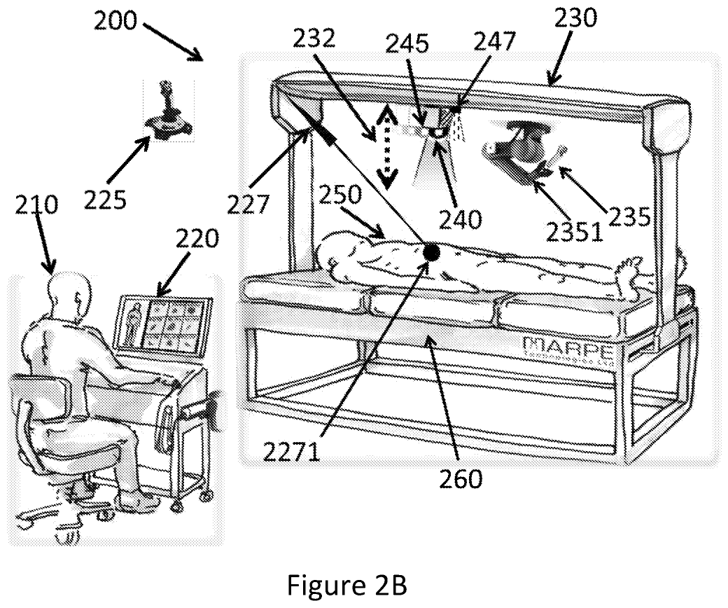

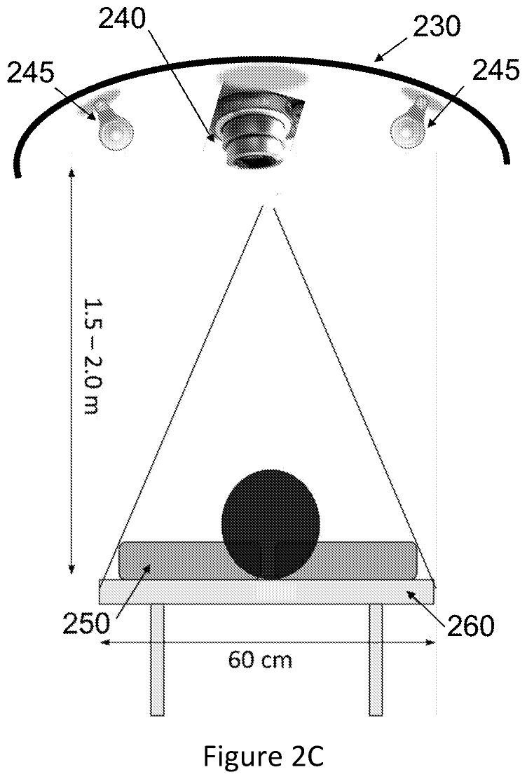

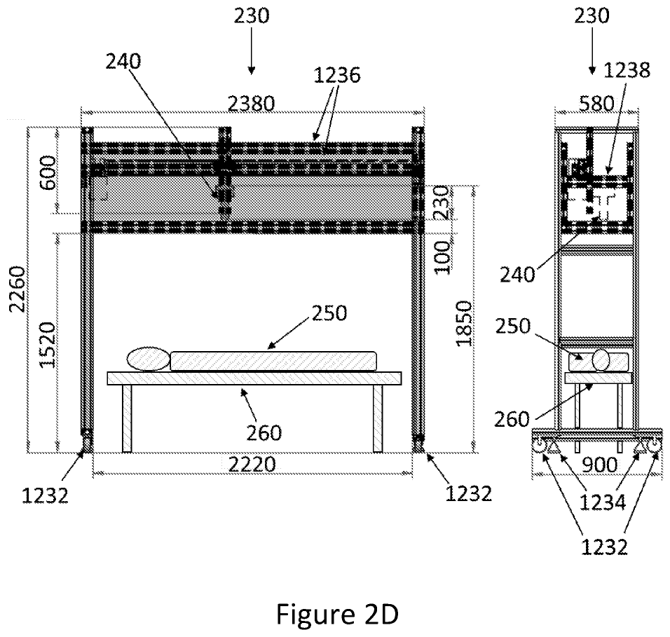

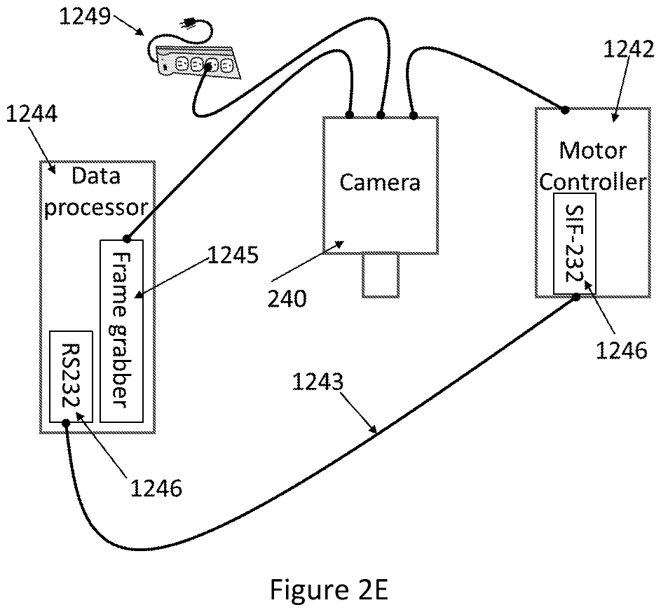

In reference to FIG. 2A-B, embodiments of the system are shown, illustrating the main features of the system (200). In FIG. 2A, the system (200) comprises a patient support system (260), in this case a bed, on which the patient (250) lies. A maneuvering mechanism (230) supports a primary imaging system (240), in this embodiment a line (1D) camera, configured to generate at least one image of a patient (250) and, optionally, at least one secondary imaging system enabled to acquire close-up images (not shown). At least one light source (245) (three are shown) ensures that the skin of the patient (250) is adequately illuminated. The primary imaging system (240) is maneuvered by the maneuvering mechanism (230), sequentially imaging the entire body. The maneuvering system can maneuver the camera or cameras in at least the Y direction (280). Maneuvering can be selected from a member of a group consisting of: moving it along the X direction (270), moving it along the Y direction (280), moving it along the Z direction (290), rotating it around the X (270) direction, rotating it around the Y (280) direction, rotating it around the Z (290) direction and any combination thereof.

FIG. 2A further illustrates the set of directions, as referred to herein. The Z direction (290) is vertical (and perpendicular to the floor). The Y direction (280) is parallel to the longitudinal axis of the patient support system (and to the longitudinal axis of the body) and parallel to the floor. The X direction (270) is perpendicular to both the Y direction and the Z direction. It is therefore parallel to the floor and parallel to the shorter side of the upper surface of the patient support system.

The system further comprises a workstation (220) comprising at least one processing system and a display capable at least of displaying images of the body and images of body moles. The workstation is capable of both automatically identifying suspicious moles and of accepting input from an operator (210), comprising at least identifying a particular mole as suspicious. The processing system controls acquisition of close-up images such that a mole of interest is automatically correlatable between at least one scanned image and the physical mole on the body of the patient (250), without need for the operator to label it.

FIG. 2B illustrates another embodiment of the system (200), comprising a patient support system (260), in this case a bed, on which the patient (250) lies. A maneuvering mechanism (230) supports a primary imaging system (240), in this embodiment a height (290) adjustable (232) line (1D) camera configured to generate at least one image of a patient (250), a 3D distance sensor (247) to accurately measure, in 3D, the distance between the primary imaging system (240) and the surface of the patient (250), and a secondary imaging system (235) enabled to acquire close-up images.

In this embodiment, the secondary imaging system (235) is a dermoscope maneuverable by a robotic arm (2351).

From the 3D distance between the primary imaging system (240) and the surface of the patient (250), a measurement of at least one dimension of the patient—the contour of the surface of the patient facing the primary imaging system (240)—can be determined.

At least one light source (245) (three are shown) ensures that the skin of the patient (250) is adequately illuminated. The primary imaging system (240) is maneuvered by the maneuvering mechanism (230), sequentially imaging the entire body. The maneuvering system can maneuver the camera or cameras in at least the Y direction (280) and the Z (290) direction. Maneuvering can be selected from a member of a group consisting of: moving it along the X direction (270), moving it along the Y direction (280), moving it along the Z direction (290), rotating it around the X (270) direction, rotating it around the Y (280) direction, rotating it around the Z (290) direction and any combination thereof.

The system comprises a workstation (220) comprising at least one processing system and a display capable at least of displaying images of the body and images of body moles. The workstation is capable of both automatically identifying suspicious moles and of accepting input from an operator (210), comprising at least identifying a particular mole as suspicious. The processing system controls acquisition of close-up images such that a mole of interest is automatically correlatable between at least one scanned image and the physical mole on the body of the patient (250), without need for the operator to label it.

The embodiment of FIG. 2B further comprises a pointer (227) maneuverable by a joystick (225). The pointer can illuminate a spot (2271) on the body of the patient (250). Maneuvering the joystick changes the location of the illuminated spot (2271). The joystick (225) is in communication with at least one processing system, so that the illuminated spot (2271) is correlated with an identifiable location on the displayed image, so that if the illuminated spot (2271) is illuminating a mole, an image of the mole is identifiable in the display. The identifiable location in the image can be identifiable by a method selected from a group consisting of: a symbol overlaying the identifiable location, a symbol surrounding the identifiable location, placing the identifiable location at a predetermined position on the screen and any combination thereof. Preferably, the predetermined location is the center of the display.

In all embodiments, the processing system, preferably but not necessarily in the workstation, comprises systems to:
a. Maneuver the 1D camera and the at least one secondary imaging system, if present, either independently or together, in at least one of the motions consisting of: move along said X direction, move along said Y direction, move along said Z direction, rotate around said X direction, rotate around said Y direction, and rotate around said Z direction
b. In some embodiments, combine images to form single images of substantially the entire surface of, preferably, one side of the body. In general, there will be one image for each of the left, right, front and back sides of the body. In these embodiments, extra images (for non-limiting example, of the armpit area) can be taken and combined with at least one of the other images.
c. Locate moles.
d. Map the location of the moles. Moles placed on the map can be those identified by the automatic location system, or they can be moles identified by a physician or technician.
e. Track mapped moles between successive images. As a patient's images change from one scan to another, due to changes in weight, age and positioning, moles will change apparent position in the image; the processing system can identify moved moles as identical to previously mapped moles.
f. Identify suspicious moles. Moles can be labeled as suspicious by the processing system, or by a physician or technician.
g. Generate close-up images of said suspicious moles, either by refocusing the 1D camera or by using at least one secondary imaging system, e.g. dermoscope.
h. Parameterize suspicious moles from either the original images of the suspicious moles, from the close-up images or, preferably, both.
i. Create a score for each mole from the parameterizations of either the original image of the suspicious mole, from the close-up image or, preferably, both. Scores can also be created manually.
j. Group moles.
k. Rank moles.
l. For each patient, create and maintain a database containing at least skin maps, mole locations, original images of moles, close-up images of moles, mole parameterizations, mole scores, mole classifications, mole groupings, physicians' notes, notes from other involved personnel, and other information as needed.
m. Ensure that the motions are properly synchronized for each camera and for the cameras relative to each other so that the scan is properly carried out, and that close-ups are taken at the proper times of the proper moles.

FIG. 2C schematically illustrates the dimensions of an embodiment of the system, wherein the system is mounted on a bed (260). In FIG. 2C, the patient (250) is lying on the bed (260), which is approximately 60 cm wide; in this embodiment, this is the effective field of view of the 1D camera of the primary imaging system (240). In some embodiments, this is the field of view of the 1D camera, in other embodiments, this is the extent in the X direction visible to the 1D camera by combining the camera's field of view and the maneuverability of the camera in the Y direction. The primary imaging system (240) is approximately 1.5-2 m above the patient. A cross-section of the maneuvering mechanism (230) is shown, as is the light source (245) (shown as two lights), also attachable to the maneuvering mechanism, used to illuminate at least a portion of the patient. The at least one light source is configured to provide sufficient light such that clear images of said body can be acquired. In preferred embodiments, it is also configured such that the light will not be bright enough to dazzle the patient.

The light source can provide homogeneous illumination of the patient, or it can provide heterogeneous illumination of the patient. A heterogeneous illumination is used to optimize contrast in the images and thereby to improve detection of moles. A non-limiting example of the use of heterogeneous illumination would be to provide increased illumination on the underside of the breast compared to the illumination over the breastbone, thereby enabling a clear image of the skin in the breast area (avoiding a dim image due to under-illuminating the partly-shaded under-breast area) while also providing a clear image in the breastbone area (avoiding a low-contrast image due to over-illuminating the unshaded breastbone area).

In the best embodiment, the camera system comprises a high resolution color 1D camera mounted on a maneuvering mechanism that enables the camera to scan an entire side of the body, thereby providing an image of that side of the body. In some embodiments, the camera is a CCD camera.

It further comprises a linear height measuring system that measures the distance between the camera and the surface of the body and creates a height map for the body. In some embodiments, measurement is via at least one sensor configured to measure this distance. The height map enables the system to adaptively focus the camera on the body surface, for rectification of the image so that focus is maintained independent of the thickness of the body in the region being imaged and to ensure accurate measurement of mole sizes. Focus can be maintained by refocusing the camera or by moving the camera in the Z direction, or both.

FIG. 2D schematically illustrates the dimensions of another embodiment of the system, wherein the system is movable on wheels (1232). In FIG. 2D, the approximate locations of the patient (250) and the bed (260) are shown in an out-of-scale manner.

In this embodiment, the primary imaging system comprises a single camera (240) which can move both in the Y direction (lengthwise to the patient) and in the X direction (perpendicular to the patient), as well as in the Z direction, as described above, to keep the distance between the patient's skin and the camera approximately constant. The system (200) is 2.26 m high, 90 cm wide and 2.38 m long. The effective width of the system, the distance between the upright supports and, therefore, the maximum widthwise travel of the camera, is 58 cm. The distance between the base of the camera and the floor is 1.52 m.

The camera (240) can be moved on rails, both longitudinally (1236) and crossways (widthwise) (1238).

The wheels (1232) that enable the system to be rolled along a floor can be seen at the corners of the bases of the uprights. The system also comprises chocks (1234) that enable the system to be fixed in position. The chocks (1234) can be raised during periods when the system is to be transported and lowered to a position where their bases are slightly below the bottoms of the wheels when the system is to be fixed in position.

An embodiment of a camera control system is shown in FIG. 2E. In this embodiment, the position of the camera (240) is alterable by a motor controller (1242), which is controlled by a data processor (1244). Power for the exemplary embodiment shown is mains power (1249); battery power or any other power source in the art can be used. In the embodiment shown, wired connection (1243) are used; in other embodiments, wireless connections or a combination of wired and wireless connections can be used. In this embodiment, RS232-SIF232 connectors (1246) are used; any wired or wireless connections known in the art that allow data transmission can be used. The data processor (1244) further comprises image acquisition software to transfer images from the camera to the processor. Any image transfer method known in the art can be used, for non-limiting example, a frame grabber (1245).

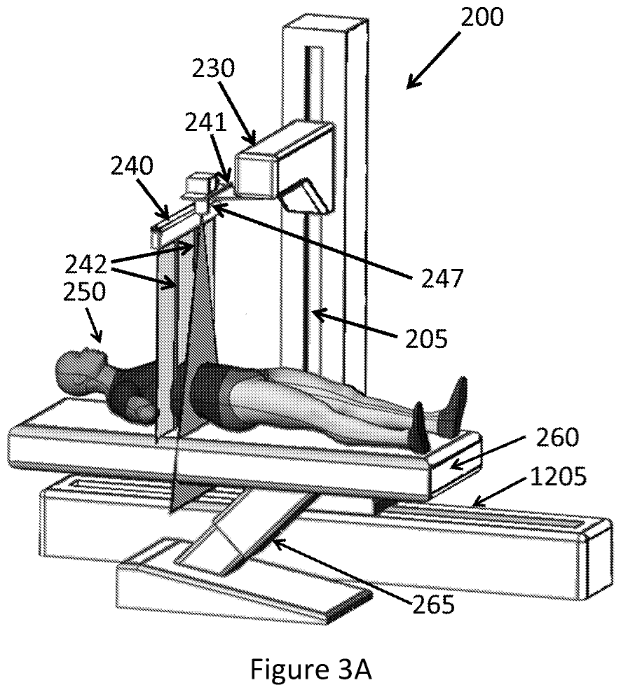

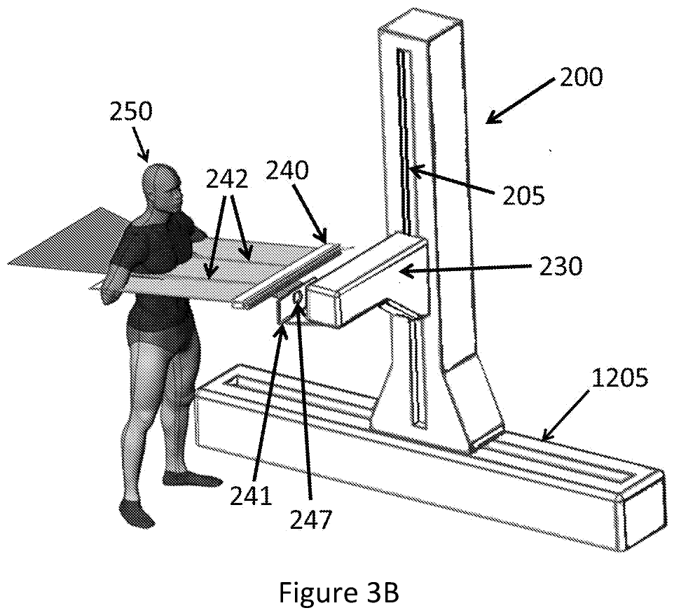

FIGS. 3A and 3B illustrate an embodiment of the system comprising a switching mechanism (241) in mechanical communication with the maneuvering mechanism (230), which enables the primary imaging system (240) and the 3D distance sensor (247) to be reversibly switched between a configuration (FIG. 3A) for imaging a patient (250) lying down and a configuration for imaging (FIG. 3B) a standing patient (250). In some embodiments, the switching mechanism (241) also enables switching of the secondary imaging system (235) between a configuration (FIG. 3A) for imaging a patient (250) lying down and a configuration for imaging (FIG. 3B) a standing patient (250).

FIG. 3A illustrates the system of the present invention (200) configured to image a patient (250) lying down (in a prone position). The switching mechanism (241) is configured to image a prone patient (250). The maneuvering mechanism (230) is in a position above the body of the patient (250), with the primary imaging system (240) and the 3D distance sensor (247) facing downward towards the patient. The widthwise extent of the patient is indicated by lines (242); preferably the widthwise extent of the patient is determinable from the 3D distance from the maneuvering mechanism (230) to the patient (250).

In the embodiment shown, a first height-adjustment mechanism (schematically indicated, 205) enables adjustment of the height of the maneuvering mechanism (230). A second height-adjustment mechanism (265) enables adjustment of the height of the bed (260). In some embodiments, the distance between the patient (250) and the primary imaging system (240) can be held substantially constant by altering at least one of the height of the maneuvering mechanism (230), the height of the primary imaging system (240) and the height of the bed (260).

A lateral adjustment mechanism (indicated schematically, 1205) enables the maneuvering mechanism (230) to be moved in the Y direction so as to scan at least a portion of the length of the patient (250).

FIG. 3B illustrates the system of the present invention (200) configured to image a patient (250) standing up (in a vertical position). The switching mechanism (241) is configured to image a standing patient (250). The maneuvering mechanism (230) is in a position to a side of the body of the patient (250), with the primary imaging system (240) and the 3D distance sensor (247) facing sideward (in the Y direction) towards the patient. The widthwise extent of the patient is indicated by lines (242); preferably the widthwise extent of the patient is determinable from the 3D distance from the maneuvering mechanism (230) to the patient (250).

In the embodiment shown, a height-adjustment mechanism (schematically indicated, 205) enables the maneuvering mechanism (230) to be moved in the Z (vertical) direction so as to scan at least a portion of the length of the patient (250).

In some embodiments, the distance between the patient (250) and the primary imaging system (240) can be held substantially constant by altering the lateral position of the maneuvering mechanism (230) by means of the lateral adjustment mechanism (1205).

In order to scan substantially the entire the body facing the camera, the camera must be maneuvered such that, during the scan, substantially every part of the body will be within the field of view of the camera at some point. To ensure that the body is imaged along substantially its entire length, the camera's field of view must traverse substantially the entire length of the body, which can be done most simply by moving the camera in the Y direction, by rotating it about an axis in the X direction, Y direction or by a combination thereof (i.e., re-orienting the same).

To ensure that the body is imaged across substantially its entire width, the camera's field of view must cover substantially the entire width of the body, which can be done either by providing a camera with field of view at least as wide as the thickest body likely to be imaged, or by moving the camera so that its field of view covers the thickest body likely to be imaged. This movement is done most simply by moving the camera in the X direction, by rotating it about an axis in the Y direction, or by a combination thereof.

The system can also comprise a linear illumination system which moves with the camera while it scans the patient's body. The illumination is automatically calibrated according to the measured heights for constant illumination conditions.

The system typically comprises a single main camera, so cannot, in general, image the all parts of the body in a single scan. Therefore, several scans are required to image substantially the entire skin surface. In practice, four scans are optimal—front, back, left and right sides. These images are joined to form a single image of substantially the entire skin surface by “stitching”—matching features near the edges of the images in order to accurately join the images. For four images, stitching is required only four times to create an image of the entire body surface; one stitching for each pair of adjoining images (front-right, right-back, back-left, left-front), to create a unique image of the entire body.

The system comprises a system to mark suspicious moles. In some embodiments, this comprises a secondary light source that marks suspicious moles by a placing a spot of light directly on the patient's body. In preferred embodiments, a mark is also placed on an image of the mole on a touch screen, so that the operator can indicate the mole of interest by touching the screen.

Suspicious moles can also be entered into the system by a physician or technician, independently or in conjunction with the automatic detection system. Methods of indicating suspicious moles include, but are not limited to: touching on a touchscreeen the image of the mole, shining a beam of light on an image of the mole on a screen, shining a beam of light on the mole on the body; entering a unique identifier for the mole; and any combination of these.

The system further comprises the patient's medical files, including, but not limited to, a database of images of the patient's body and of the patient's moles, the operator's notes, instructions to the patient, actions taken in regard to the patient, results of those actions, and recommended follow-up.

The system is designed to facilitate fast and effective diagnosis. A patient's file includes huge amounts of data: very large total body images, many close-up images of mole, mole parameters and the patient's medical information. The amount of data increases rapidly, as each skin test adds a new set. Therefore, sophisticated tools are required to display effectively and to manage this huge amount of data.

In addition, moles are spread over the entire surface of the body, increasing the sophistication needed in the management and display system. In this system, the management and display system includes:
a. Integrated viewers for the total body images. By pointing at a specific mole, all the relevant mole data, including historical data, is displayed
b. Integrated viewers for close-up images, so that the operator can see, on demand, all close-ups of a given mole
c. Automatic calculation of mole morphology and color parameters—the ABCDE parameters, which are:
   i. Measurements of the mole: Asymmetry, Border shape, and Diameter.
   ii. The mole's Color map.
   iii. Display of the mole's Evolution over time. (Evolution of a given mole and identification of new moles)
d. Integrated viewers for registered and normalized images from periodic tests.
e. Fast display of moles sorted by parameters and global scores.
f. Display of the Mole's Color map to enhance visualization.
g. Comparison of suspicious moles with mole databases.
h. Statistics tools.
i. An ability to create instructions to technician for further image acquisition.

For each suspicious mole, the operator, preferably a skilled technician or dermatologist, points a dedicated device, for example a dermoscopy microscope, at the suspicious mole to capture a detailed image of the highlighted mole. Close-up image acquisition is computer controlled so that no mole is missed and so that all close-up images are permanently and uniquely correlated to the original mole of which they are close up images so that, for example, the location on the body of the mole shown in close-up is known and can be easily shown.

Close up images are taken immediately after each of the scans of a side of the body, while the patient is lying in the same position as that of scan, in order to best ensure that the mole and its close up images are correlated, as described above.

A wide-angle camera ensures that, while taking close-up images of a mole, small movements of patient do not affect the positioning information, which could result in imaging the wrong area, or in a blurred image.

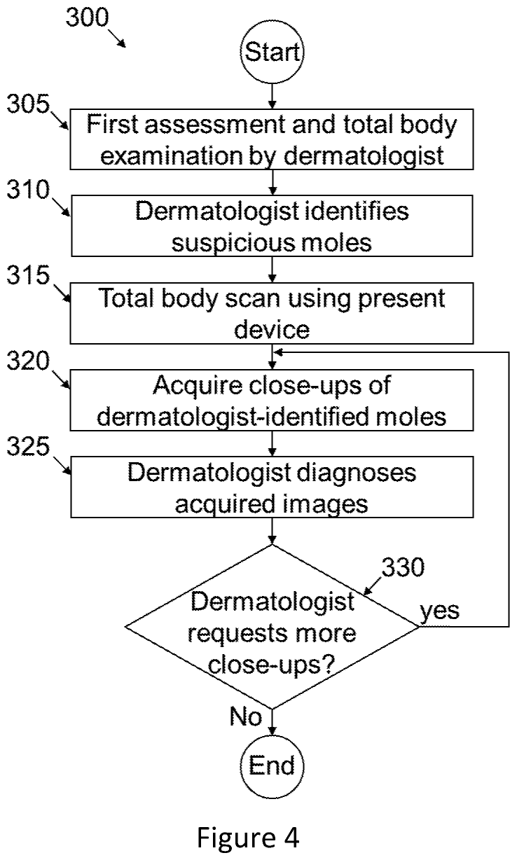

In reference to FIGS. 4-7, flow charts are shown of an embodiment of the method (300-600) for automatic mapping, tracking and evaluation of moles. FIG. 4 shows an embodiment of the process of the initial evaluation. In the initial evaluation, the dermatologist assesses the patient and carries out a total body scan (305), identifying suspicious moles (310). A technician then carries out a total body scan, using the present device (315), acquiring close-ups of the suspicious moles identified by the dermatologist (320). The dermatologist then diagnoses the acquired images (325). If the dermatologist requests more close-ups as a result of the diagnosis (330), further close-ups are acquired and, if necessary, further diagnosis is carried out.

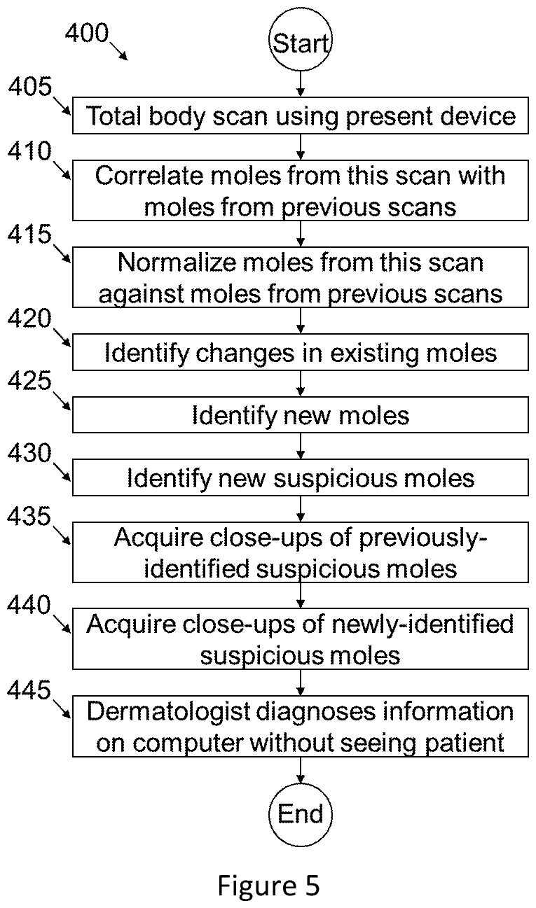

A set of periodic scans will then be carried out by the technician, without the dermatologist being present. The frequency of the scans and the number of scans so carried out will be decided on by the dermatologist during the initial assessment. A flow chart of an embodiment of the procedure for carrying out a computerized periodic scan by a technician is shown in FIG. 5. The process starts with a total body scan using the present device (405). The processor then correlates moles from this scan with moles from previous scans (410) and normalizes the moles from this scan against moles from previous scans (415), identifies changes in existing moles (420) and identifies new moles (425).

As the patient's body images will change from one scan to another, due to changes in the patient's weight and age (for example, skin quality changes with age) and due to changes in the patient's position (for example, is the patient oriented in exactly the same way), correlation of moles from the current scan against moles from previous scans includes finding the location of the mole on the image of the body can correlating the location of this mole against the locations of moles in previous scans. Because of apparent changes in the mole's position (due to changes in position) and real changes in the location of the mole (due to changes in weight and age), correlation (i.e., registration) includes correlation by position relative to the image of the body and correlation by means of triangulation and other registration methods of the mole against known features of the body (including other moles). It can also include correlation by mole shape or mole score.

Furthermore, in the present invention, the comparison between scans is done at image level. The same mole is located in several images using image processing technics. For example similarity of the surrounding of the mole in two different scan is looked for in order to ensure the same mole is disclosed. The XYZ axis data is also used for the coarse registration so as to facilitate in the comparison.

From analysis of the changes in existing moles and analysis of the new moles, the system identifies new suspicious moles (430) and acquires close-ups of previously-identified suspicious moles (435) and new suspicious moles (440). These data are sent to the dermatologist, who refines his diagnosis without the need to see the patient.

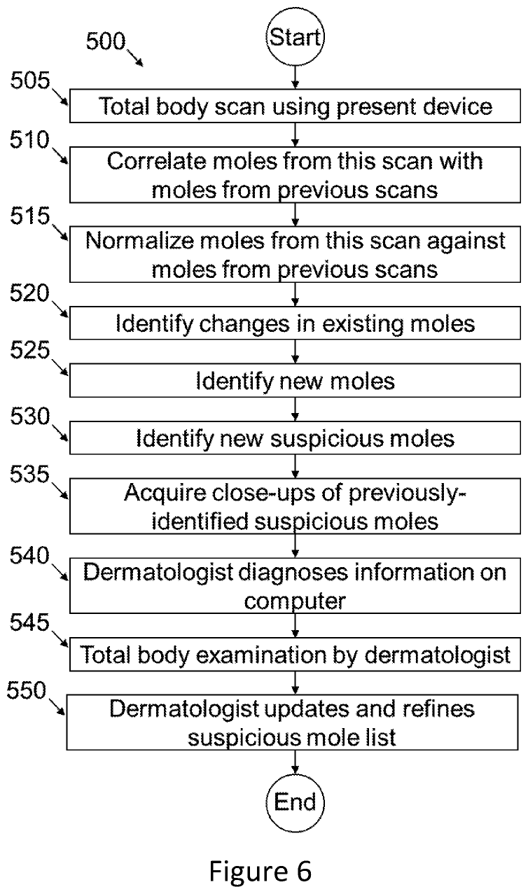

After the set of periodic scans is complete, another examination will be carried out where the dermatologist examines the patient. A flow chart of an embodiment of this examination is shown in FIG. 6. A total body scan is carried out by the technician, as was done for the computerized periodic scan. The processor then correlates moles from this scan with moles from previous scans (510) and normalizes the moles from this scan against moles from previous scans (515), identifies changes in existing moles (520) and identifies new moles (525) and new suspicious moles (530). Close-ups of previously-identified suspicious moles (535) are acquired. The dermatologist then refines his diagnosis using the computerized information (540) and carries out a total body examination of the patient (545). Using both the computerized information and the information acquired from the personal examination, the suspicious mole list is updated and refined (550).

The set of periodic scans followed by a personal examination can be repeated for as long as is needed.

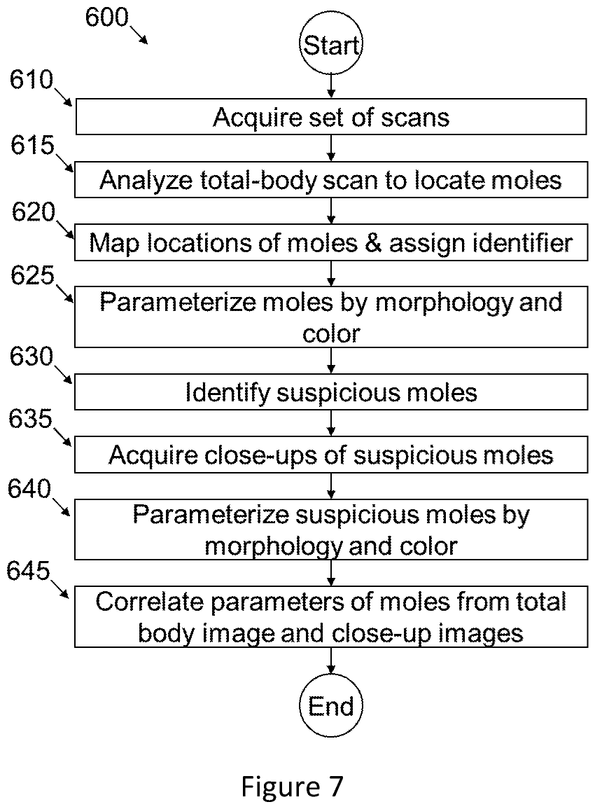

In reference to FIG. 7, a flow chart is shown of an embodiment of a method (600) for automatic mapping, tracking and evaluation of moles. The set of scans is acquired (610), the images are analyzed to locate the moles (615) in the total body image, locations of the moles are mapped (620) and each mole is given an identifier by which it can be uniquely identified. The moles are parameterized (625) by morphology and color to identify suspicious moles (630). Close-up images are acquired of suspicious moles (635) and the close-up images are parameterized by morphology and color (640). The mole images from the total-body images and the close-up images are correlated by their parameters to uniquely link close-up images to the corresponding mole in the total body image (645).

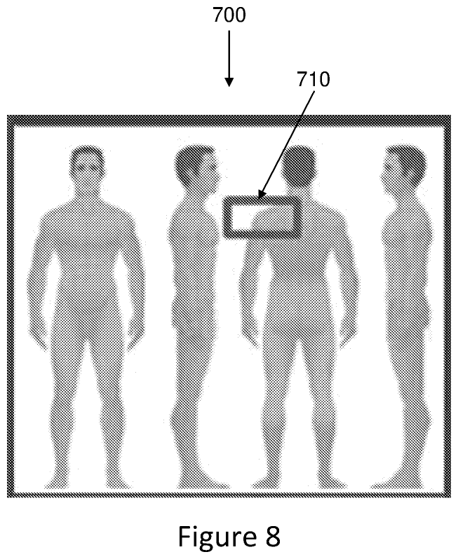

In reference to FIG. 8, an image is shown of a schematic of the four sides of the body (700), as it appears on the screen in some embodiments of the system. The square 710 illustrates an area of the body of interest; the screen will display in close up an image of the part of the body included within the square 710.

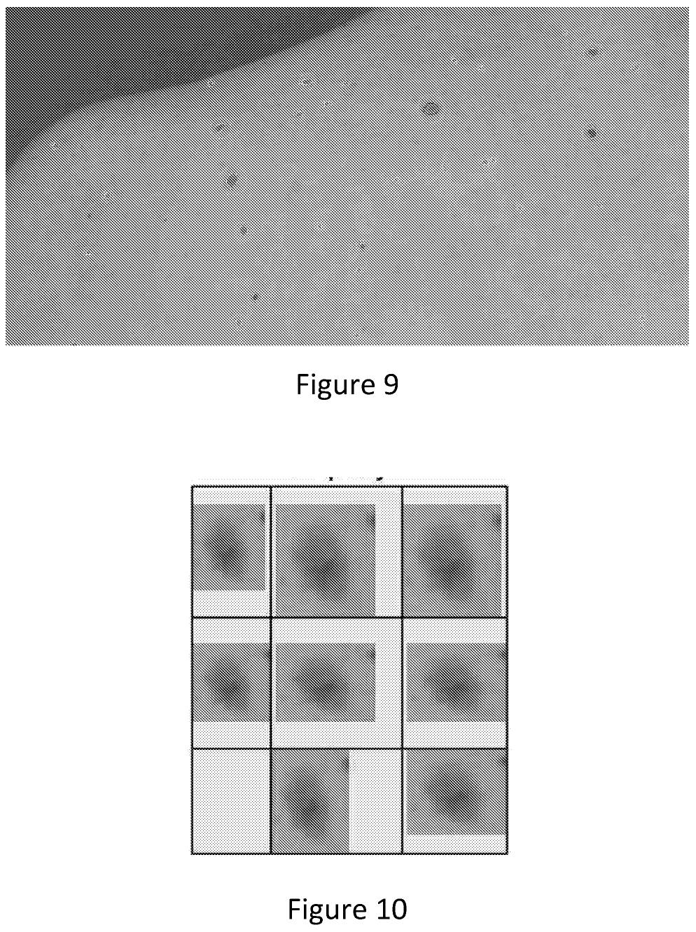

In reference to FIG. 9, an image is shown of a patient's back in the area indicated by the square 710, showing the output of the mole location software. Identified moles are shown circled in green. It can be seen all moles have been identified; scars and freckles have been ignored. Furthermore, any suspicious mole is further identified.

According to another embodiment of the present invention, all moles are identified and marked, while suspicious moles are marked in a different manner (different color, different identification).

According to another embodiment, the physician can mark a mole as suspicious (even if the system does not identify said mole as suspicious.

In reference to FIG. 10, images are shown of a set of moles with similar scores. The moles can easily be checked at a glance, or compared with moles in a mole database.

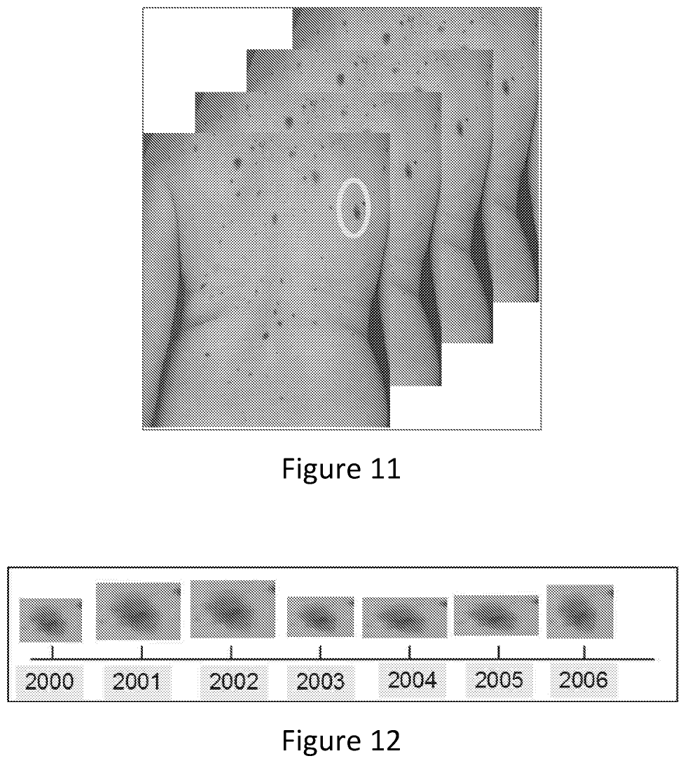

In reference to FIG. 11, images are shown of a portion of a total body image over time. The circled mole is an example of a suspicious mole.

In reference to FIG. 12, images are shown of the evolution of a mole such as the circled mole in FIG. 11 over time. The mole can be quickly and efficiently checked for changes over time.

An advantage of the present system is that, by computerizing the process and by uniquely identifying each mole so that its location on a skin map is known, and since the examination is primarily visual, examinations of moles can be done remotely, for example, over the web. For example, the technician can carry out a periodic examination at a nurse's office, while the dermatologist can examine the results in his office, at home or at any other location by using wireless devices. He can easily send mole images to experts for remote consulting. It also becomes feasible to train paramedical personnel as “mole testers”. An example of a person quickly trainable as a mole tester is a retired nurse. Such a person could be trained to recognize suspicious moles. An appropriate web application could dispatch images of moles and lesions to a secure computer available to the “mole tester”. Lesions are then classified and scored by the “moles tester”, using computer-aided tools. Moles are then sorted by their scores, increasing the efficiency of the diagnosis by the dermatologist.

Since mole classification is subjective, especially in cases where the ambiguity is large, the same moles can be sent to several mole testers, with the final score for a given mole being generated from the individual scores. Any method known in the art can be used to generate the final score, such as, but not limited to, simple averaging, weighted averaging, or a voting process.

Once moles are scored, they can be grouped, with moles of similar scores being assigned to the same group, or being assigned to groups such that each mole belongs to a group with a similar score, but the groups are limited in size. If limited-size groups are used, a group size of less than approximately 20 is preferred.

Moles can also be ranked, with moles with higher scores being ranked higher, as “more suspicious”.

On average, there are around 100 lesions and moles on the surface of a human body. Therefore, the number of moles to be examined during mass screening of a large population is huge. To make “mole testers” work efficiently, there is a need for dedicated productivity tools. One example of such a tool is an automated tool to automatically group moles with close scores and to display to “mole testers” groups of moles. If the “mole tester's” scores agree with the automated scores, then one glance and one click and the group is classified. By using this and similar productivity tools, the overall process of mass screening is significantly enhanced.

Due to privacy concerns, in additional to only transmitting data using the web security measures known in the art, total body images will not be sent via the web; only the small regions containing individual moles will be sent.

In the present device, a single 1D camera is employed, in a scanning mode, in place of the sets of 2D cameras in devices of the prior art. The advantages of a single 1D camera are:
1. It reduces distortion. There is perspective distortion only in one direction, so there is better image quality and a more precise estimation of the dimensions of small moles.
2. There are fewer limits on the size of the body. Human bodies vary significantly in both length and width. With a single scanning camera, the maximum mappable length of a body is limited only by the maneuvering length of the maneuvering mechanism in the Y direction and the width of the body only by the width of the camera's effective field of view.
3. The photographic process is simple and short. Since only small portion of the body is imaged at any one time, the system is tolerant to small movements by the patient.
4. Camera registration and orientation is simpler than with a set of 2D cameras; the system is designed so that the maneuvering system provides registration between successive scan lines,; whereas in a set of 2D cameras, the operator is required to maintain an overlapping area
5.Camera registration and orientation is simpler that with a set of 2D camera. In a set of 2D cameras is required to orient each camera while maintaining overlapping areas between each pair of cameras.
6. Image registration is not needed as no joins are made between images of the different sides of the body, instead of the many joins required with a multi-camera device.
7. Adaptive and precise focusing of the lens is possible while the camera is moving. The camera can be moved and refocused as it moves along the body, thereby attaining an optimum focus for each slice of the body, no matter the difference in thickness between different slices of the body; human bodies are not flat. The linear height system enables the adaptive focusing.
8. There is no waste of processing resources. There are only small, if any, frame overlaps with line scanning, which eliminates redundant data that waste processing resources, particularly in high-speed, high-resolution applications. Speed is crucial since the system needs to locate suspicious moles while taking photographs so the technician can immediately acquire close-up images of moles. Moreover, when the image size is huge (Giga pixels), as it will be for high-resolution scans, then speed is important
9. The patient needs not to be totally nude while being photographed, as only a body portion about 2-3 cm wide is required to be uncovered at any one time while the camera is moving.
10. The lighting system is simple and of low power—more friendly to the patient and less dazzling.
11. The camera is hung over the bed as a bridge, so the footprint of the device is no larger than the bed over which it sits.

One difficulty with prior art mole detection systems is body hair; it can be difficult to identify, form a photograph, whether a feature is hair or a mole. In the present device, the system comprises a distinguishing system to assist the processing system to distinguish between hairs and moles. In some embodiments, the system comprises an infrared camera that assists the system in distinguishing between hair and skin. In other embodiments, the distinguishing system comprises a hair alignment system wherein the hairs are made to occupy the minimum possible area in the images. In the best of these embodiments, this is accomplished by making the hairs stand perpendicular to the skin. In some embodiment where the hairs occupy the minimum possible area, a static electric field is used to ensure that hairs stand perpendicular to the skin. In other embodiments where the hairs occupy the minimum possible area an air suction device performs the same function.

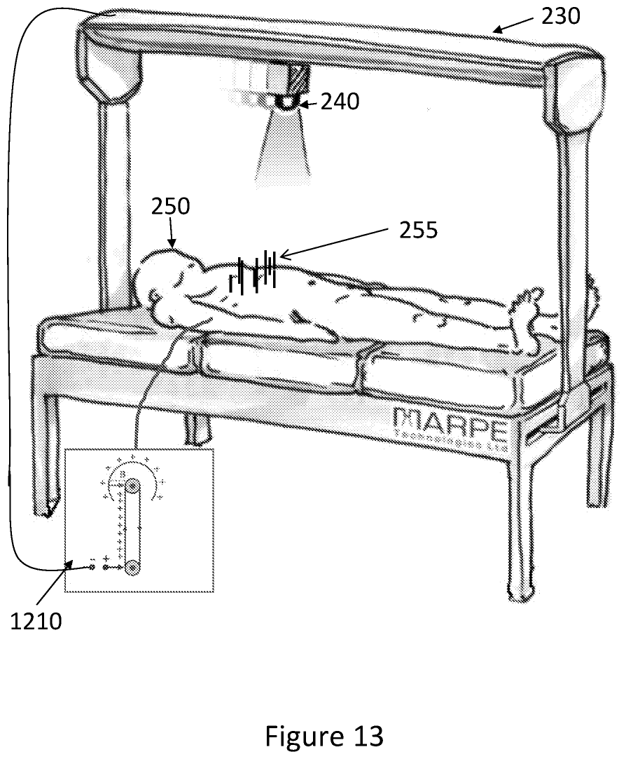

In some embodiments, the hair alignment system aligns the hairs on the whole surface of the body facing the camera. In other embodiments, movement of the hair alignment mechanism is synchronized with movement of the maneuvering system so that the hair alignment mechanism aligns hairs only in portions of the body being imaged. In embodiments of the hair alignment mechanism comprising a static electric field, the field is generated by a van de Graaf generator, Pelletron generator or other generator of static electricity known in the art, as shown schematically in FIG. 13. In FIG. 13, the patient (250) has hair (255) on his chest. One pole of the van de Graaf generator (or other generator of static electricity) (1210) is in contact with the patient. The other pole of the generator is in contact with the maneuvering mechanism (230). Therefore there is a strong vertical electric field between the patient (250) and the maneuvering mechanism (230), which will cause the patient's hairs to align themselves parallel to the electric field, in this case vertically, causing the hairs to appear in the images as small circular dots, clearly distinguishable from moles, and too small to interfere with the identification or classification of the moles.

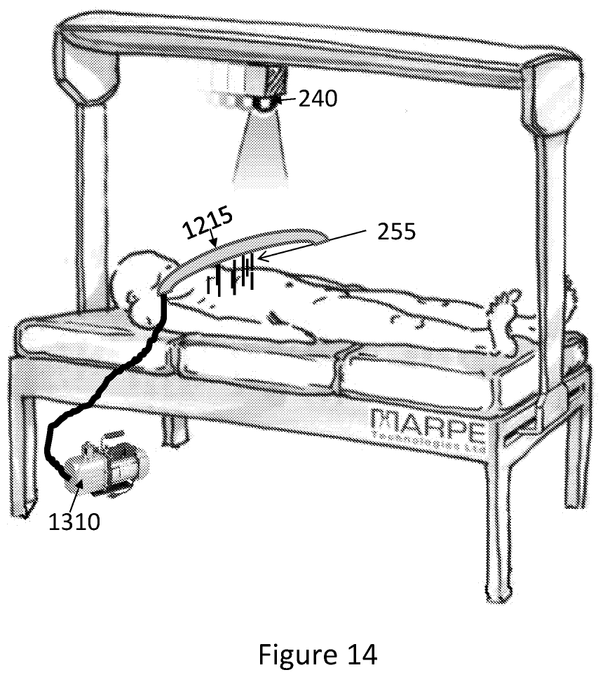

In embodiments of the hair alignment mechanism comprising an air suction device, the air suction device (FIG. 14) comprises a suction pump (1310) attached to a vent (1315) movable with the camera (240) (not shown). The vent provides sufficient suction that the hairs (255) in the skin in the portion being imaged are vertical, causing the hairs (255) to appear in the images as small circular dots, clearly distinguishable from moles, and too small to interfere with the identification or classification of the moles.

As disclosed hereinabove, it is only necessary to have a 2-3 cm wide portion of the patient exposed at any time during imaging. Therefore, the patient's body can be covered, except for a 2-3 cm wide portion moving with the maneuvering mechanism, thereby enabling the patient to keep warm, as is not possible with devices of the prior art where several 2D cameras are used. Having the body covered considerably increases the patient's comfort; only the absolute minimum of the patient need be exposed, and that for the minimum possible time, thereby enabling the patient to keep warm and also considerably reducing mental discomfort due to being unclothed. In addition, in embodiments where the covering means is draped somewhat above the patient, exposed parts will be visible only from above and will therefore be visible only with difficulty to persons in the room with the patient, further increasing the patient's comfort.

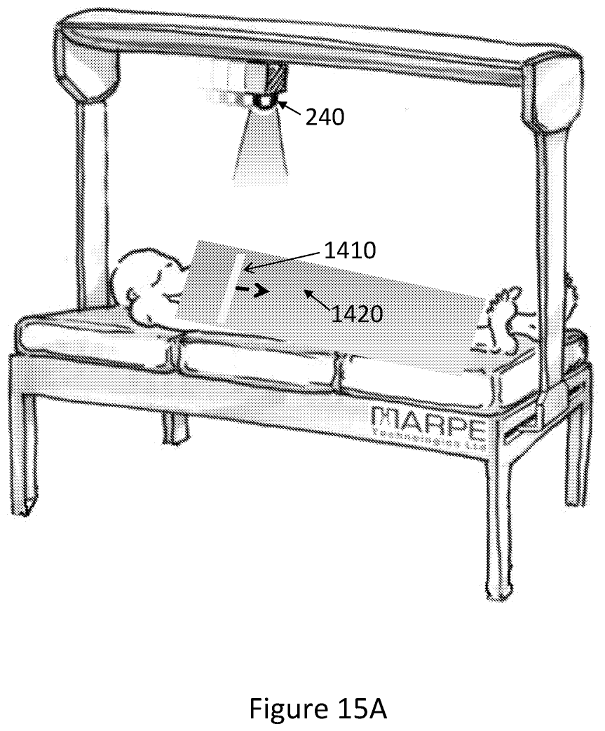

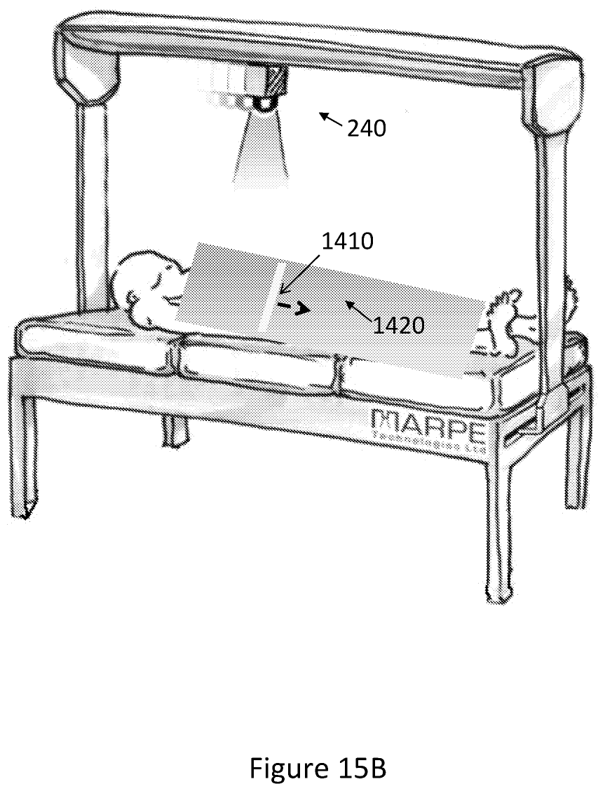

Preferred embodiments of the present system comprise such an exposing mechanism. A non-limiting example of an embodiment of an exposing mechanism providing such a covering means is schematically illustrated in FIG. 15A-B. The exposing mechanism (1420) is shown covering the body, except for an exposure slit (1410). FIG. 15A shows the system during an early part of the scan. The camera (240) is near the top of the body and the exposure slit (1410), below the camera is also near the top of the body, exposing only the portion of the body in the field of view of the camera. The dashed arrow shows the direction of motion of the exposure slit. FIG. 15B shows the system during a later part of the scan. The field of view of the camera (240) has moved; the position of the exposure slit (1410) has also moved, so that the body remains substantially covered, except for the portion of the body in the field of view of the camera.

In embodiments where the exposure slit is substantially the same width as the body, the exposure slit is maneuvered only in the Y direction.

In other embodiments, the exposure slit is substantially narrower than the width of the body. In such embodiments, the exposure slit is maneuvered in both the X and Y directions.

In some embodiments, the hair alignment mechanism aligns all the hair on at least the side of the body being imaged. In some variants of these embodiments, the hair on at least the side of the body being imaged is aligned previous to the start of the imaging of that side of the body. In other variants, the alignment of the hair on at least the side of the body being imaged starts substantially simultaneously with the start of imaging. In all variants, the hair on each portion of the body remains aligned at least until that portion of the body has been imaged.

In some embodiments, the hair alignment mechanism aligns only a portion of the hair, the minimum portion being the hair on the portion of skin being imaged. In such embodiments, there are two variants. In one, the portion of the hair is aligned before the start of imaging of that portion of skin. In the other, alignment occurs substantially simultaneously with the start of imaging of that portion of the skin.

1.-104. (canceled)

105. A system for mapping and tracking at least one mole on a surface of a body, comprising:
at least one optical system configured to scan a surface of said body and to generate at least one image of at least a portion of said surface, said optical system comprising at least one primary imaging device attachable to at least one first maneuvering mechanism, said at least one image comprising substantially all of said portion of said surface facing said at least one primary imaging device, said at least one mole identifiable from said at least one image; and
at least one switching mechanism in mechanical communication with said at least one optical system,
wherein said at least one switching mechanism is configured to reversibly switch said at least one optical system between generating said at least one image of a standing patient and generating said at least one image of a patient lying down.

106. The system of claim 105, additionally comprising at least one measurement device configured to make at least one three dimensional (3D) measurement of at least one dimension of said body.

107. The system of claim 105, additionally comprising at least one secondary imaging device attachable to at least one second maneuvering mechanism, said second maneuvering mechanism configured to automatically aim said secondary imaging device at said at least one mole, said secondary imaging device thereupon being configured to automatically capture at least one image of said at least one mole, wherein, by correlation between a scanned image comprising an image of at least one image mole and at least one 3D measurement of at least one corresponding body mole corresponding to said at least one image mole, said secondary imaging device is accurately aimable at said at least one corresponding body mole.

108. The system of claim 105, additionally comprising at least one bi-directional light pointer in communication with an indicating mechanism, said bi-directional light pointer having at least two modes, said at least two modes selected from a group consisting of: a display-to-body mode, a body-to-display mode and any combination thereof, wherein, in said body-to-display mode, at least one body mole on said surface being selected by manually pointing said light pointer at said at least one body mole, an indication is provided for a corresponding at least one display mole in a displayed image; and in said display-to-body mode, at least one displayed mole in a displayed image being selected using an indicating mechanism in communication with a display, light from said light pointer is shined on a corresponding at least one body mole on said surface.

109. The system of claim 105, additionally comprising a hair alignment mechanism configured to orient said hair substantially perpendicular to said surface of said body facing said at least one primary imaging device, to minimize apparent size of said hair in said image, wherein a start of alignment of said hair is either simultaneous with a start of said imaging of at least a portion of said body's surface or before a start of said imaging of at least a portion of said body's surface, said alignment of said hair continuing during said imaging of said at least a portion of said body's surface.

110. The system of claim 105, wherein said first maneuvering mechanism can maneuver said at least one primary imaging device so as to cause it to move in a manner selected from a group consisting of: moving in the X direction, moving in the Y direction, moving in the Z direction, rotating about an axis in the X direction, rotating about an axis in the Y direction, rotating about an axis in the Z direction, and any combination thereof, and said at least one primary imaging device is configured for one or more in a group consisting of
keeping in focus said surface of said body facing said at least one primary imaging device by at least one of: maneuvering said at least one primary imaging device in said Z direction and refocusing said at least one primary imaging device;
acquiring images in said X direction of substantially the entire said surface of said body facing said at least one primary imaging device by at least one of: moving said at least one primary imaging device in at least said X direction, rotating said at least one primary imaging device about said Y direction, and providing a at least one primary imaging device with a field of view at least as wide as said surface of said body facing said at least one primary imaging device; and
acquiring images in said Y direction of substantially the entire said surface of said body facing said at least one primary imaging device by at least one of: maneuvering said at least one primary imaging device in said Y direction, and rotating said at least one primary imaging device about said X direction.

111. The system of claim 105, additionally comprising an alignment synchronization mechanism configured to synchronize movement of said alignment mechanism and said maneuvering mechanism such that said hair alignment mechanism aligns hairs in portions of said body being imaged.

112. The system of claim 105, wherein said first maneuvering mechanism additionally comprises at least one light source configured to illuminate at least a portion of said body, said at least one light source configured to provide sufficient light such that clear images of said body can be acquired; and said at least one light source is configured to illuminate said at least a portion of said body's surface in a manner selected from a group consisting of: a homogeneous manner and a heterogeneous manner; wherein, for said heterogeneous manner of illumination, the difference between the illumination of at least one part of said illuminated portion of said body's surface and at least one other part of said illuminated portion of said body's surface being configured to optimize contrast in said image.

113. The system of claim 105, wherein said system additionally comprises at least one processing system in communication with said at least one primary imaging device, comprising a program of machine-readable instructions embodied on a computer readable memory and executable by a digital data processor, configured for executing one or more in a group consisting of
combining said at least one image to form a single image of substantially the entire surface of said body;
locating in said at least one image said at least one mole;
mapping a location of said at least one mole on said surface;
creating at least one image of said at least one mole; and
identifying, from said at least one mole, a set of suspicious moles, said set being empty if no suspicious mole is present on said surface.

114. The system of claim 107, further comprising at least one processing system in communication with said at least one secondary imaging device, wherein at least one secondary imaging device is further configured to generate, said set of suspicious moles not being empty, at least one close-up image of at least one member of said set of suspicious moles; said at least one processing system comprises a program of machine-readable instructions embodied on a computer readable memory and executable by a digital data processor, configured to instruct at least one of a group consisting of: said at least one primary imaging device and said at least one secondary imaging system to move in a manner selected from a group consisting of: move along said X direction, move along said Y direction, move along said Z direction, rotate around said X direction, rotate around said Y direction, and rotate around said Z direction.

115. The system of claim 105, wherein said body can be re-imaged periodically and for each of said at least one mole, at least one image of said at least one mole generated at least one first time is compared to at least one image of said at least one mole generated at least one second time, from said comparison the evolution of said at least one mole over time being trackable.

116. The system of claim 105, additionally comprising a distinguishing system for enabling distinguishing between hair and skin, said distinguishing system comprising at least one of: an infrared camera, a static electric field and air suction.

117. The system of claim 105, wherein said at least one mole is analyzable according to ABCDE parameters, said parameters comprising: (i) measuring one mole according to its (A)symmetry, (B)order shape, (C)olor and (D)iameter, and (ii) displaying said mole's history over a series of said images over time, (E)volution of said at least one mole being tracked and of any new mole being identified and tracked.

118. The system of claim 105, wherein, said set of suspicious moles not being empty, said at least one suspicious mole is parameterizable from at least one image selected from a group consisting of: an image of said at least one mole, a close-up image of said at least one mole, and any combination thereof; for each said at least one suspicious mole, said parameterization creates a score for each said at least one suspicious mole; and said at least one suspicious mole is grouped or ranked according to said score.

119. The system of claim 105, wherein at least one of said measurement device and a height measuring system in communication with said at least one primary imaging device and said tracking system is configured to maintain at least one of a focal distance of said at least one primary imaging device and a distance between said at least one primary imaging device and said body, maintaining said portion of said body being imaged in focus independent of a thickness of said body at a location of said portion of said body.

120. The system of claim 105, additionally comprising an exposing mechanism configured to expose at least partially a section of said body to said at least one primary imaging device; and an exposure synchronization mechanism configured to synchronize movement of said exposing mechanism and said first maneuvering mechanism such that said at least one primary imaging device images only said exposed section.

121. A method for mapping and tracking at least one mole on a surface of a body, comprising steps of:
providing a system for mapping and tracking at least one mole on a surface of a body, comprising:
at least one optical system configured to scan a surface of said body and to generate at least one image of at least a portion of said surface, said optical system comprising at least one primary imaging device attachable to at least one first maneuvering mechanism, said at least one image comprising substantially all of said portion of said surface facing said at least one primary imaging device, said at least one mole identifiable from said at least one image; and
at least one switching mechanism in mechanical communication with said at least one optical system;
attaching said at least one primary imaging device to said first maneuvering mechanism;
sequentially imaging substantially all of the surface of said body facing said at least one primary imaging device in at least one image by maneuvering said first maneuvering mechanism so as to move said at least one primary imaging device along said surface of said body facing said at least one primary imaging device;
thereby reversibly switching, via said at least one switching mechanism, between generating said at least one image of a standing patient and generating said at least one image of a patient lying down.

122. The method of claim 121, additionally comprising a step of providing at least one measurement device configured to make at least one 3D measurement of at least one dimension of said body.

123. The method of claim 121, additionally comprising steps of providing at least one secondary imaging device attachable to at least one second maneuvering mechanism, said second maneuvering mechanism configured to automatically aim said secondary imaging device at said at least one mole, said secondary imaging device thereupon being configured to automatically capture at least one image of said at least one mole; and correlating between a scanned image comprising an image of at least one image mole and at least one 3D measurement of at least one corresponding body mole corresponding to said at least one image mole; and accurately aiming, by use of said correlation, said secondary imaging device at said at least one corresponding body mole.

124. The method of claim 121, additionally comprising steps of providing at least one bidirectional light pointer in communication with an indicating mechanism, said bidirectional light pointer having at least two modes, said at least two modes selected from a group consisting of: a display-to-body mode, a body-to-display mode and any combination thereof; and selecting, in said display-to-body mode, at least one displayed mole in a displayed image using an indicating mechanism in communication with a display, and of shining light from said light pointer on a corresponding at least one body mole on said surface.
